(19) United States
(12) Patent Application Publication
(10) Pub. No.: US 2024/0299701 A1
(43) Pub. Date: Sep. 12, 2024

(54) CATHETER SYSTEMS AND METHODS OF USE

(71) Applicant: Piraeus Medical, Inc., Rochester, MN (US)

(72) Inventor: Jared Hutar, Cromwell, MN (US)

(21) Appl. No.: 18/667,470

(22) Filed: May 17, 2024

Related U.S. Application Data
(63) Continuation-in-part of application No. 18/314,082, filed on May 8, 2023.
Application No. 18/314,082 is a continuation-in-part of application No. 18/064,843, filed on Dec. 12, 2022.
(60) Provisional application No. 63/340,276, filed on May 10, 2022.
(60) Provisional application No. 63/289,038, filed on Dec. 13, 2021.
(60) Provisional application No. 63/340,276, filed on May 10, 2022.

(51) Int. Cl. A61M 25/00; A61M 25/01

(52) U.S. Cl. CPC A61M 25/0012; A61M 25/0023; A61M 25/0045; A61M 25/005; A61M 25/0097; A61M 25/01; A61M 2025/0004; A61M 2025/0046; A61M 2025/0062; A61M 2205/0222; A61M 2205/0238; A61M 2207/10

(57) ABSTRACT

The disclosure is directed to a catheter system comprising an inner catheter and/or an outer catheter configured to at least partially receive the inner catheter. In some embodiments, the inner catheter comprises a proximal hub, a distal portion, and/or a pusher wire extending between the proximal hub and the distal portion. In some embodiments, the inner and/or the outer catheter does not include a smooth inner surface. In some embodiments, the inner and/or outer catheter includes an inner surface comprising a series of recessed surfaces. In some embodiments, the recessed surfaces are formed by the spaces between the catheter's reinforcement structure, where the spaces are filled and/or sealed by an encasement sleeve. In some embodiments, the reinforcement structure forms support surfaces for structures traversing through the inner diameter. In some embodiments, at least a portion of the support surfaces and/or recessed surfaces include reflown polymer and/or a hydrophilic coating.

### CROSS-REFERENCE TO RELATED APPLICATIONS

The present application is a Continuation-in-part of U.S. patent application Ser. No. 18/314,082, filed on May 8, 2023, which is a Continuation-in-part of U.S. patent application Ser. No. 18/064,843, filed on Dec. 12, 2022, which claims priority to U.S. Provisional Patent Application No. 63/340,276, filed May 10, 2022, and claims priority to U.S. Provisional Patent Application No. 63/289,038, filed on Dec. 13, 2021; and U.S. patent application Ser. No. 18/314,082 claims priority to U.S. Provisional Patent Application No. 63/340,276, filed May 10, 2022, each of which is incorporated herein by reference in its entirety.

### BACKGROUND

The disclosure generally relates to medical devices and methods of use. Some embodiments of the system described herein include devices and methods for performing thrombectomy or embolectomy in a patient. Acute Ischemic Stroke (AIS) can be caused by thrombus, embolus, or other occlusions in regions of the internal carotid artery (ICA) such as the Petrous part, Cavernous part, or Cerebral part. Approaches for performing thrombectomy or embolectomy to treat AIS include positioning a device-such as an aspiration catheter, a balloon guiding catheter, or other devices—in the carotid artery at a location upstream from the occlusion, typically at a proximal location in the artery such as the cervical part. Navigation to the proximal location can be difficult due to the tortuous nature and small vessel size of the vasculature usually involved in an AIS.

Traditional approaches for treating AIS and lesions in other areas of the body take a more significant amount of time as the physician uses trial and error to determine which device/combination of devices will reach and remove the occlusion. In a situation where “time is brain,” reducing the amount of time required to remove the thrombus, embolus, or other occlusion is crucial to achieving the best possible outcome for the patient.

Therefore, there is a need for improved devices and methods for mechanical revascularization such as thrombectomy and embolectomy in the ICA and other vasculature. In particular, there is a need for systems and methods that provide enhanced efficacy and efficiency of treatment.

### SUMMARY

In some embodiments, the disclosure is directed to a catheter system comprising an outer catheter having a proximal end, a distal end located opposite the proximal end, a working lumen extending between the proximal end and the distal end, an outer surface defining an outer diameter, and an inner surface defining an inner diameter. In some embodiments, the catheter system includes an inner catheter having a proximal hub, a distal portion having a distal end located opposite the proximal hub, an outer surface defining an outer diameter, an inner surface defining an inner diameter, and a pusher wire extending between the proximal hub and the distal portion. In some embodiments, the working lumen of the outer catheter is configured to at least partially receive the inner catheter. In some embodiments, one or more of the outer catheter and inner catheter comprise an outer surface that is smoother than an inner surface. Some embodiments are directed to a kit comprising one or more of the outer catheter and inner catheter.

In some embodiments, the distal portion of the inner and/or outer catheter comprises a hypotube. In some embodiments, the hypotube may comprise a distal portion and a proximal portion located opposite the distal portion, wherein the proximal portion may be configured to taper to a proximal end coupled to a pusher wire. In some embodiments, the pusher wire is configured to facilitate navigation of the inner catheter through the working lumen of the outer catheter.

In some embodiments, the hypotube comprises an inner surface and an outer surface. In some embodiments, the outer surface may be covered with a heatshrink material. In some embodiments, at least a portion of the heatshrink material and at least a portion of the inner surface of the hypotube are coated with a lubricious coating. The lubricious coating, in some embodiments, comprises one or more of a hydrophilic coating and silicone. In some embodiments, the outer surface may be covered with a reflown polymer material. In some embodiments, at least a portion of the reflown polymer material and at least a portion of the inner surface of the hypotube are coated with a lubricious coating. In some embodiments, the lubricious coating may comprise one of a hydrophilic coating and silicone. In some embodiments, the hypotube comprises a stainless steel hypotube. In some embodiments, the hypotube comprises a nitinol hypotube. In some embodiments, the hypotube may comprise a combination of stainless steel and nitinol materials. In some embodiments, the hypotube comprises a laser-cut hypotube. In some embodiments, the hypotube includes a length of about 10 to 30 cm, where about 20 centimeters is sufficient for most applications.

In some embodiments, the outer catheter comprises a device wall, and the inner catheter comprises a device wall, wherein the device wall of the outer catheter includes at least one polymer coupled to an outer catheter reinforcement structure, and wherein the device wall of the inner catheter includes at least one polymer coupled to an inner catheter reinforcement structure. The outer catheter reinforcement structure may comprise a braid and/or coil reinforcement structure, and the inner catheter reinforcement structure may comprise a braid and/or coil reinforcement structure. In some embodiments, at least a portion of the braid and/or coil structure forms support surfaces of a traversing structure (e.g., guide wire, catheter)

In some embodiments, the device wall of the outer catheter may be located between a first hydrophilic coating and a second hydrophilic coating, and the device wall of the inner catheter may be located between a third hydrophilic coating and a fourth hydrophilic coating. In some embodiments, the first hydrophilic coating is located on the outer surface of the outer catheter and is configured to reduce surface friction and increase lubricity between the outer surface of the outer catheter and a vessel wall. In some embodiments, the first hydrophilic coating may comprise a substantially smooth surface. In some embodiments, the second hydrophilic coating may be located on the inner surface of the outer catheter and may be configured to reduce surface friction and increase lubricity between the inner surface of the outer catheter and the outer surface of the inner catheter. In some embodiments, the second hydrophilic coating may comprise a textured surface. In some embodiments, the third hydrophilic coating is located on the outer surface of the inner catheter and configured to reduce surface friction and increase lubricity between the inner surface of the outer catheter and the outer surface of the inner catheter. In some embodiments, the third hydrophilic coating comprises a substantially smooth surface. In some embodiments, the fourth hydrophilic coating may be located on the inner surface of the inner catheter and may be configured to reduce surface friction and increase lubricity on the inner surface of the inner catheter. In some embodiments, the fourth hydrophilic coating may comprise a textured surface.

In some embodiments, the pusher wire comprises a round wire. In some embodiments, the pusher wire comprises a flat wire.

In some embodiments, the outer catheter may comprise a device wall including at least one polymer coupled to an outer catheter reinforcement structure. In some embodiments, the outer catheter reinforcement structure includes a braid and/or coil reinforcement structure. In some embodiments, the device wall of the outer catheter may be located between a first hydrophilic coating and a second hydrophilic coating. In some embodiments, the first hydrophilic coating is located on the outer surface of the outer catheter and is configured to reduce surface friction and increase lubricity between the outer surface of the outer catheter and a vessel wall. In some embodiments, the first hydrophilic coating comprises a substantially smooth surface. In some embodiments, the second hydrophilic coating may be located on the inner surface of the outer catheter and may be configured to reduce surface friction and increase lubricity between the inner surface of the outer catheter and the outer surface of the inner catheter. In some embodiments, the second hydrophilic coating may comprise a textured surface.

In some embodiments, the system includes an outer catheter having a proximal end, a distal end located opposite the proximal end, a working lumen extending between the proximal end and the distal end, an outer surface defining an outer diameter, and an inner surface defining an inner diameter. In some embodiments, the catheter system includes an inner catheter having a proximal end and a distal end located opposite the proximal end, wherein the working lumen is configured to at least partially receive the inner catheter, and wherein the inner catheter comprises a hypotube. In some embodiments, the system includes a kit comprising one or more of an inner catheter, outer catheter, and a pusher wire.

In some embodiments, the hypotube comprises an inner surface and an outer surface. In some embodiments, the outer surface may be covered with a heatshrink material. In some embodiments, at least a portion of the heatshrink material and at least a portion of the inner surface of the hypotube are coated with a lubricious coating. In some embodiments, the lubricious coating comprises one of a hydrophilic coating and silicone. In some embodiments, the outer surface is be covered with a reflown polymer material. In some embodiments, at least a portion of the reflown polymer material and at least a portion of the inner surface of the hypotube are coated with a lubricious coating. The lubricious coating may comprise one of a hydrophilic coating and silicone.

In some embodiments, the hypotube may comprise a stainless steel hypotube. In some embodiments, the hypotube comprises a nitinol hypotube. In some embodiments, the hypotube may comprise a combination of stainless steel and nitinol materials. In some embodiments, the hypotube comprises a laser-cut hypotube.

In some embodiments, the outer catheter comprises a device wall including at least one polymer coupled to an outer catheter reinforcement structure. In some embodiments, the outer catheter reinforcement structure includes a braid and/or coil reinforcement structure. In some embodiments, the device wall of the outer catheter may be located between a first hydrophilic coating and a second hydrophilic coating. In some embodiments, the first hydrophilic coating is located on the outer surface of the outer catheter and is configured to reduce surface friction and increase lubricity between the outer surface of the outer catheter and a vessel wall. In some embodiments, the first hydrophilic coating may comprise a substantially smooth surface. In some embodiments, the second hydrophilic coating may be located on the inner surface of the outer catheter and may be configured to reduce surface friction and increase lubricity between the inner surface of the outer catheter and an outer surface of the inner catheter. In some embodiments, the second hydrophilic coating comprises a textured surface. In some embodiments, the second hydrophilic coating comprises a smooth surface.

In some embodiments, the inner and/or outer catheter includes an inner diameter, a reinforcement structure, an encasement sleeve, and/or an outer diameter. In some embodiments, the reinforcement structure is configured to at least partially form a shape of the inner diameter along a length of the catheter. In some embodiments, the encasement sleeve is configured to encase at least a portion of the reinforcement structure. In some embodiments, the encasement sleeve is configured to prevent a fluid flow from the inner diameter of the catheter to the outer diameter of the catheter.

In some embodiments, the reinforcement structure includes gaps between reinforcement surfaces along the inner diameter. In some embodiments, the reinforcement surfaces include the portion of the reinforcement structure orthogonal to a center point of the inner diameter. In some embodiments, the reinforcement structure is configured to define one or more shapes along the inner diameter of the catheter. In some embodiments, an inner diameter varies along at least a portion of the length of the inner diameter. In some embodiments, a distance between the inner diameter and the outer diameter varies along at least a portion of the length of the catheter.

In some embodiments, the catheter does not comprise Polytetrafluoroethylene. In some embodiments, the catheter does not comprise a Polytetrafluoroethylene liner along the inner diameter. In some embodiments, an outer diameter surface varies less than an inner diameter surface. In some embodiments, an outer diameter surface is smoother than an inner diameter surface. In some embodiments, the reinforcement structure includes a coil structure. In some embodiments, the inner and/or outer catheter includes a hub. In some embodiments, at least a portion of an inner diameter of the hub is coated with a hydrophilic substance.

In some embodiments, a method of manufacturing the inner and/or outer catheter comprises one or more steps. Some embodiments include a step of forming or placing a reinforcement structure on a mandrel. Some embodiments include a step of placing at least one encasement sleeve over the reinforcement structure, which may include one or more polymers described herein. Some embodiments include a step of coupling the reinforcement structure and the at least one polymer together to create a catheter shaft. Some embodiments include a step of removing the catheter shaft from the mandrel.

In some embodiments, an outer surface of the catheter shaft is smoother than an inner surface of the catheter shaft after it is removed from the mandrel. In some embodiments, the inner surface includes peaks formed by the reinforcement structure. In some embodiments, the inner surface includes valleys formed by the at least one polymer. In some embodiments, the reinforcement structure includes hollow portions. In some embodiments, valleys are formed by a portion of the at least one polymer exposed between the hollow portions.

Some embodiments include a step of coupling a hub to a proximal end of an inner and/or outer catheter shaft. Some embodiments include a step of coating an inner diameter of the hub with a hydrophilic substance. Some embodiments include a step of coating an inner diameter of the catheter shaft and an inner diameter of the hub with a hydrophilic substance.

In some embodiments, the reinforcement structure includes hollow portions configured to impart flexibility to the catheter shaft. In some embodiments, a distal bending force required to bend the catheter shaft more than 1200 at a distal region furthest from a hub attachment area is 15% or less of a proximal bending force required to bend the catheter shaft more than 120° at a proximal region closest from the hub. In some embodiments, at least a portion of the catheter shaft is configured to bend to an inner radius of curvature less than 2 mm without kinking.

The foregoing, and other features and advantages of the system, will be apparent from the following detailed description according to some embodiments.

### BRIEF DESCRIPTION OF THE DRAWINGS

These and other features, aspects, and advantages according to some embodiments are described below with reference to the drawings, which are intended to illustrate, but not to limit, the system and methods of the disclosure. In the drawings, like characters denote corresponding features consistently throughout some embodiments.

FIG. 1 illustrates a perspective view of a catheter system including an outer catheter and an inner catheter, according to some embodiments.

FIGS. 2A and 2B illustrate a catheter system including an outer catheter and an inner catheter, according to some embodiments.

FIG. 3 illustrates a cross-section of an outer catheter, according to some embodiments.

FIG. 4 illustrates a cross-section of an inner catheter, according to some embodiments.

FIG. 5 illustrates a cross-section of a catheter system, including an outer catheter and an inner catheter, according to some embodiments.

FIG. 6 illustrates an outer catheter, according to some embodiments.

FIG. 7 illustrates an inner catheter, according to some embodiments.

FIG. 8 illustrates an outer catheter including various elements, according to some embodiments.

FIG. 9 illustrates a catheter system including an outer catheter and an inner catheter, according to some embodiments.

FIG. 10 illustrates an inner catheter including a hypotube, according to some embodiments.

FIG. 11 illustrates an inner catheter including a coil structure, according to some embodiments.

FIG. 12 illustrates an inner catheter including a braid structure, according to some embodiments.

FIGS. 13A and 13B illustrate cross-sectional views of a hypotube, according to some embodiments.

FIG. 14 illustrates a catheter system including an outer catheter and an inner catheter, according to some embodiments.

FIG. 15 illustrates an inner catheter comprising a hypotube, according to some embodiments.

FIGS. 16A and 16B illustrate cross-sectional views of a hypotube, according to some embodiments.

FIGS. 17 and 18 illustrate flow charts explaining the processes of applying hydrophilic coatings, according to some embodiments.

FIG. 19 shows results for a 3-point bending test performed on conventional catheters and system catheters according to some embodiments.

FIG. 20 illustrates a catheter kink test for a system versus a conventional catheter according to some embodiments.

FIG. 21 shows data for inner diameter lubricity in a simulated anatomy model according to some embodiments.

FIG. 22 shows a measured catheter distal looped compression force according to some embodiments.

FIG. 23 shows a cross-sectional view of the catheter 300, according to some embodiments.

### COMPONENT INDEX

10—catheter system
12—outer catheter
14—proximal end (outer catheter)
16—distal end (outer catheter)
18—outer surface (outer catheter)
20—inner surface (outer catheter)
22—outer diameter (outer catheter)
24—inner diameter (outer catheter)
26—inner catheter
28—proximal end (inner catheter)
30—distal end (inner catheter)
32—outer surface (inner catheter)
34—inner surface (inner catheter)
36—outer diameter (inner catheter)
38—inner diameter (inner catheter)
40a—first hydrophilic coating
40b—second hydrophilic coating
40c—third hydrophilic coating
40d—fourth hydrophilic coating
42—device wall of the outer catheter
44—device wall of the inner catheter
46—at least one polymer
48—outer catheter reinforcement structure
50—inner catheter reinforcement structure
52—hub (outer catheter)
54—hub (inner catheter)
100—catheter system
102—outer catheter
104—proximal end (outer catheter)
106—distal end (outer catheter)
108—working lumen
110—inner catheter
112—proximal hub (inner catheter)
114—distal portion (inner catheter)
116—distal end (inner catheter)
118—pusher wire
120—hypotube
122—coil structure
124—braid structure
126—distal portion (hypotube)
128—proximal portion (hypotube)
130—proximal end (hypotube)
132—inner surface (hypotube)
134—outer surface (hypotube)
136—heatshrink material
138—reflown polymer
140—lubricious coating
200—catheter system
202—outer catheter
204—proximal end (outer catheter)
206—distal end (outer catheter)
208—working lumen (outer catheter)
210—inner catheter
212—proximal end (inner catheter)
214—distal end (inner catheter)
216—hypotube
218—inner surface (hypotube)
220—outer surface (hypotube)
222—heatshrink material
224—reflown polymer
226—lubricious coating
300—inner and/or outer catheter
301—coil structure
302—recessed surface
303—contacting surface
304—thin hydrophilic coating
305—thick hydrophilic coating
306—braid
307—inner diameter
308—outer surface
310—encasement sleeve
311—outer diameter

### DETAILED DESCRIPTION

Although some embodiments and examples are disclosed below, the subject matter extends beyond the some disclosed embodiments to other alternative embodiments and/or uses, and to modifications and equivalents thereof. Thus, the scope of the claims appended hereto is not limited to some embodiments described below. For example, in any method or process disclosed herein, the acts or operations of the method or process may be performed in any suitable sequence and are not necessarily limited to any particular disclosed sequence according to some embodiments. Various operations may be described as multiple discrete operations in turn, in a manner that may be helpful in understanding some embodiments; however, the order of description should not be construed to imply that these operations are order-dependent. Additionally, the structures, systems, and/or devices described herein according to some embodiments may be embodied as integrated components or as separate components.

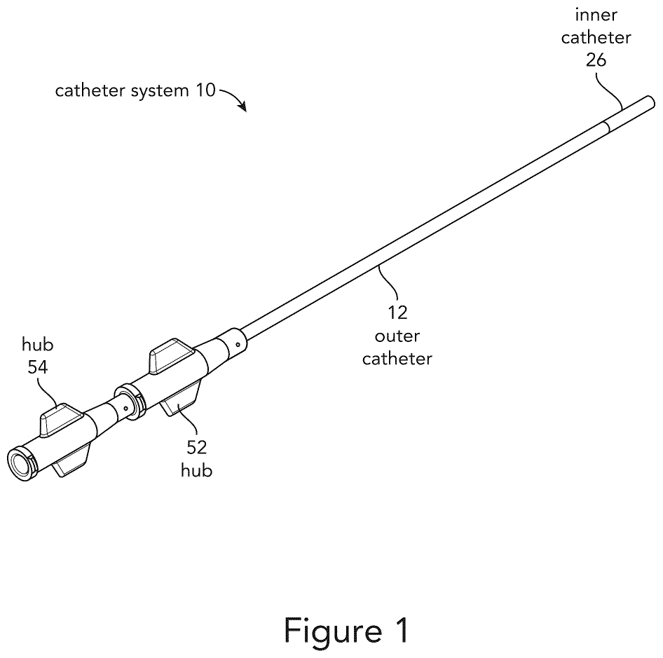

FIG. 1 illustrates a perspective view of a catheter system 10, according to some embodiments. In some embodiments, the catheter system includes an outer catheter 12 and an inner catheter 26, as illustrated in FIG. 1. In some embodiments, the outer catheter 12 includes a hub 52, and the inner catheter 26 includes a hub 54. The catheter system 10 will be described in greater detail throughout this disclosure.

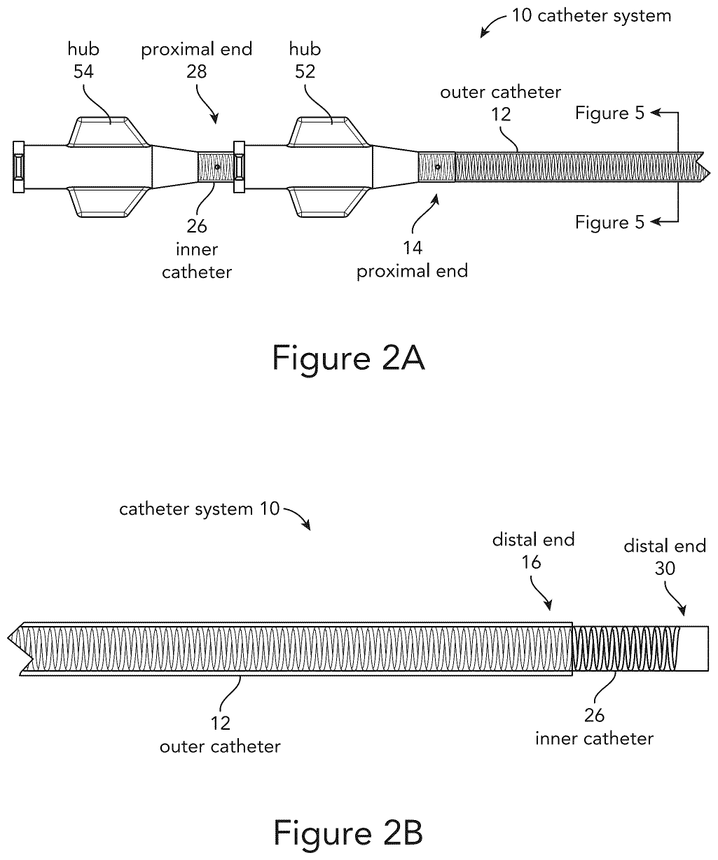

FIGS. 2A and 2B further illustrate the catheter system 10, according to some embodiments. In some embodiments, the catheter system includes an outer catheter 12 and an inner catheter 26, as illustrated in FIGS. 2A and 2B. In some embodiments, the outer catheter 12 includes a hub 52, and the inner catheter 26 includes a hub 54. As shown in FIG. 2A, the hub 52 may be located at the proximal end 14 of the outer catheter 12, and the hub 54 may be located at the proximal end 28 of the inner catheter 26. In some embodiments, the outer catheter 12 includes a distal end 16 located opposite the proximal end 14, and the inner catheter 26 includes a distal end 30 located opposite the proximal end 28. In some embodiments, the outer catheter 12 and/or inner catheter 26 includes an outer surface that is smoother than an inner surface.

In some embodiments, the outer catheter 12 may be sized and configured to at least partially receive the inner catheter 26, as illustrated in FIGS. 2A and 2B. In some embodiments, the outer catheter 12 may also be sized and configured to receive one or more traversing structures, such as a guidewire, a microcatheter, an intermediate catheter, and/or a stent retriever, to name a few non-limiting examples. In some embodiments, the catheter system 10 includes a combination of an inner and outer device (i.e., the inner and outer catheters 26, 12) that can be used concentrically, with the inner device inside of the outer device. In some embodiments, the outer and/or inner catheters 12, 26 can be used individually. For example, during a procedure, such as a thrombectomy, the outer catheter 12 may be inserted into the patient first in an initial attempt to track the outer catheter 12 distally within the anatomy to a surface of a clot. If the outer catheter 12 successfully tracks the surface of the clot, an aspiration force may be applied to the outer catheter 12, thereby removing the clot through the outer catheter 12. In some embodiments, if the outer catheter 12 is unsuccessful in tracking to the surface of the clot, the outer catheter 12 may still serve as a guide or support catheter to help deliver the inner catheter 26 through the outer catheter 12 to the surface of the clot.

In some embodiments, a method of using only a single device (i.e., the outer catheter 12) to remove the clot allows for procedures to be more efficient than current procedure practices, which often involve several steps of introducing and removing several devices. In some embodiments, the catheter system 10, including the inner catheter 26 and outer catheter 12, allows for patient anatomy to drive the procedure, rather than following the same steps for every patient, as is the current practice.

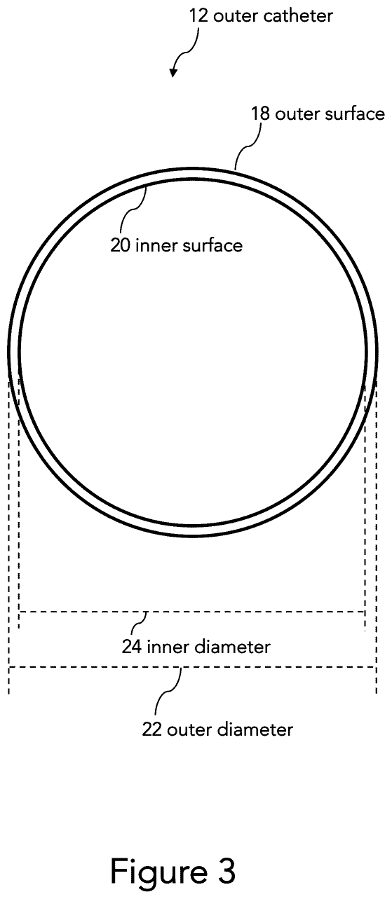

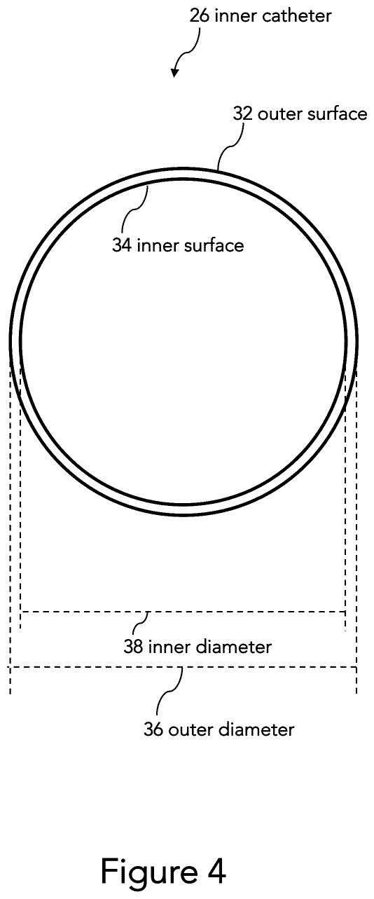

FIGS. 3 and 4 illustrate cross-section views of the outer catheter 12 and inner catheter 26, respectively. In some embodiments, the outer catheter 12 may include an outer surface 18 defining an outer diameter 22 and an inner surface 20 defining an inner diameter 24. The outer diameter 22 and inner diameter 24 may each define a broad range of dimensions, including, for example, 0.111 inches for the outer diameter 22 and 0.100 inches for the inner diameter 24, according to some embodiments. As illustrated in FIG. 4, in some embodiments, the inner catheter 26 may also include an outer surface 32 defining an outer diameter 36 and an inner surface 34 defining an inner diameter 38. In some embodiments, the outer diameter 36 of the outer surface 32 defines a measurement of 0.098 inches, and the inner diameter 38 of the inner surface 34 defines a measurement of 0.088 inches, although any inner and/or outer measurement can be used, as the dimensions listed here, for both the outer catheter 12 and the inner catheter 26, are included as non-limiting examples. In some embodiments, the outer catheter 12 and inner catheter 26 may both define a wide range of dimensions not explicitly listed in this disclosure. For example, the outer catheter 12 and inner catheter 26 may define outer and/or inner diameter dimensions between 0.003 inches and 0.18 inches, according to some embodiments.

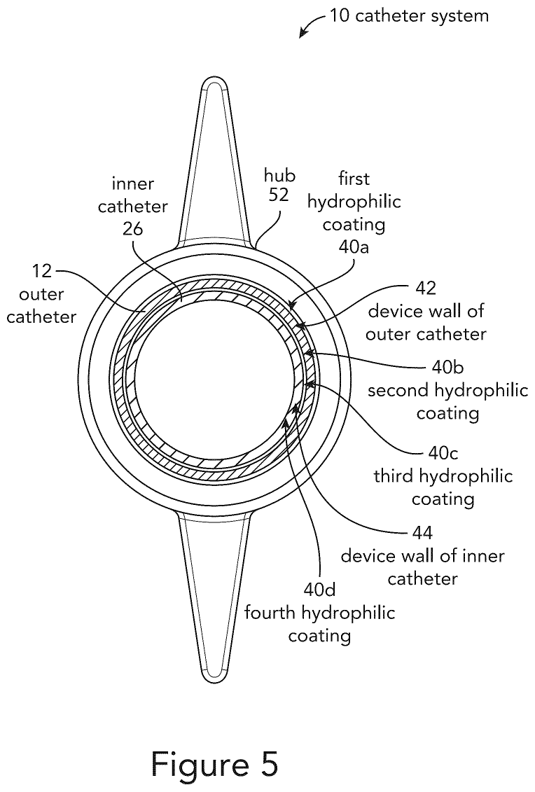

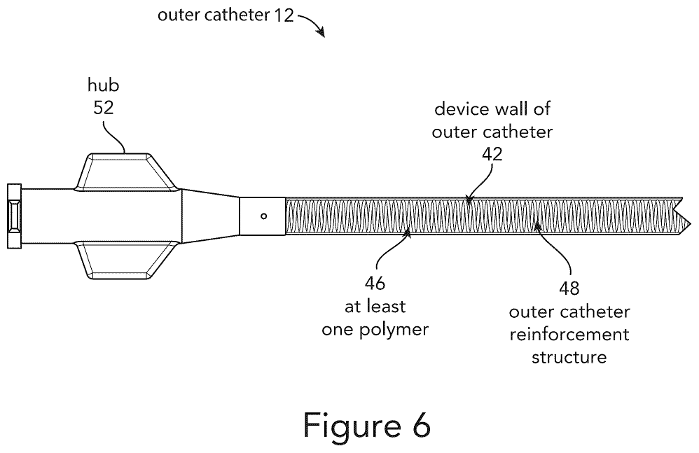

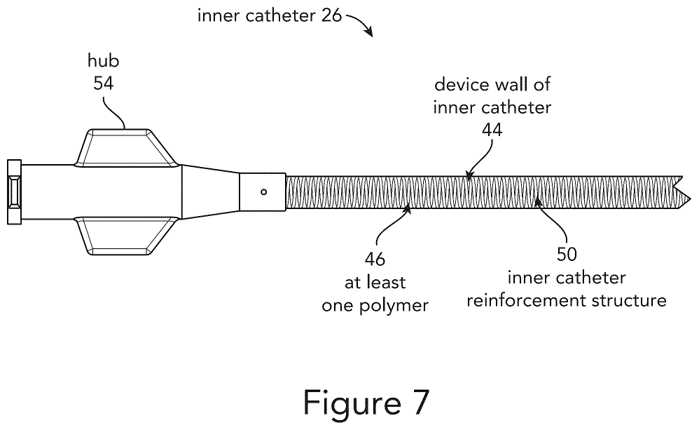

As indicated in FIG. 2A, FIG. 5 illustrates a cross-section of the catheter system 10, including both the outer catheter 12 and inner catheter 26, as well as the hub 52 of the outer catheter 12 according to some embodiments. In some embodiments, as shown in FIG. 5, the outer catheter 12 comprises a device wall of the outer catheter 42, and the inner catheter 26 comprises a device wall of the inner catheter 44. The device walls 42, 44 will be discussed further with reference to FIGS. 6 and 7. In some embodiments, the catheter system 10 may also include a first hydrophilic coating 40a, a second hydrophilic coating 40b, a third hydrophilic coating 40c, and a fourth hydrophilic coating 40d, as shown in FIG. 5.

In some embodiments, the first hydrophilic coating 40a is located on the outer surface 18 of the outer catheter 12, and the second hydrophilic coating 40b is located on the inner surface 20 of the outer catheter 12. In some embodiments, the device wall of the outer catheter 42 is located between the first hydrophilic coating 40a and the second hydrophilic coating 40b. In some embodiments, the third hydrophilic coating 40c is located on the outer surface 32 of the inner catheter 26, and/or the fourth hydrophilic coating 40d is located on the inner surface 34 of the inner catheter 26. In some embodiments, the device wall of the inner catheter 44 is located between the third hydrophilic coating 40c and the fourth hydrophilic coating 40d. In some embodiments, each of the first, second, third, and fourth hydrophilic coatings 40a-d may extend along a surface extending between the proximal ends 14, 28 and distal ends 16, 30 of the outer and inner catheters 12, 26. In some embodiments, the surface extends substantially an entire length of the catheters 12, 26. In some embodiments, the surface may extend less than a full length, such as 50%, 25%, or 10% of the entire length. In some embodiments, each of the hydrophilic coatings 40a-d is configured to cover a distalmost portion, such as 15 centimeters, of the outer and inner catheters 12, 26. It should be noted that, in some embodiments each of the hydrophilic coatings 40a-d is configured to cover any size portion of the catheters 12, 26. It should also be noted that each of the hydrophilic coatings 40a-d does not necessarily define the same length, though they may each define the same length according to some embodiments.

In some embodiments, each of the first hydrophilic coating 40a, second hydrophilic coating 40b, third hydrophilic coating 40c, and fourth hydrophilic coating 40d may comprise the same material and thickness. In some embodiments, the thickness of each hydrophilic coating 40a-d is between 0.0001 and 0.001 inches. The term “hydrophilic coating” is a species of lubricious coatings that reduce friction and increases trackability of the outer and inner catheters 12, 26, as they move within vessels and/or within one another (e.g., the inner catheter 26 moving within the outer catheter 12) according to some embodiments. Some nonlimiting examples of lubricious coatings according to some embodiments include hydrophilic coatings, silicone coatings, PTFE dust, and any other suitable lubricants. In some embodiments, coating the device wall 42, 44 with one or more lubricious coatings (e.g., hydrophilic coating 40a-d) allows the device walls 42, 44 to be thinner than traditional device walls while also improving the performance of the catheters 12, 26. In some embodiments, the device walls 42, 44 may include a thickness between 0.001 and 0.04 inches.

Referring now to FIG. 6, the outer catheter 12 is shown, as previously discussed, the outer catheter 12 includes a device wall 42 according to some embodiments. In some embodiments, as illustrated in FIG. 6, the device wall 42 comprises at least one polymer 46 and an outer catheter reinforcement structure 48. In some embodiments, the at least one polymer 46 may also be referred to as a “polymer jacket structure.” In some embodiments, the at least one polymer 46 is configured to provide at least one of flexibility and structural support to the outer catheter 12 and/or the inner catheter 26. In some embodiments, the outer catheter reinforcement structure 48 may comprise a (metallic) braid and/or coil structure, with the at least one polymer 46 filling any space within the braid and/or coil structure. In some embodiments, the at least one polymer 46 may also cover the outer catheter reinforcement structure 48. In some embodiments, the outer catheter reinforcement structure 48 is configured to provide stiffness to a proximal portion of the outer catheter 12 and flexibility to a distal portion of the outer catheter 12. In some embodiments, the amount, coil tightness, and/or composition of the outer catheter reinforcement structure 48 and/or inner catheter 26 may vary depending on the location along the length of the outer catheter 12 and/or length of inner catheter 26.

Similar to FIG. 6, FIG. 7 shows the inner catheter 26 including the device wall 44 comprising the at least one polymer 46 and the inner catheter reinforcement structure 50 according to some embodiments. In some embodiments, the inner catheter reinforcement structure 50 is substantially similar in construction to the outer catheter reinforcement structure 48, including the structure immersed in the at least one polymer 46. In some embodiments, the inner catheter reinforcement structure 50 may comprises one or both of a coil and braded structure. In some embodiments, the inner catheter reinforcement structure 50 is configured to provide stiffness to a proximal portion of the inner catheter 26, and flexibility to a distal portion of the inner catheter 26. In some embodiments, the amount, coil tightness, and/or composition of the inner catheter reinforcement structure 50 may vary depending on the location along the length of the inner catheter 26. In some embodiments, the device wall of the outer catheter 42 and the device wall of the inner catheter 44 is substantially the same construction and may comprise the same type of polymer(s) in the at least one polymer 46, as well as the same type of braid and/or coil structure in the outer catheter reinforcement structure 48 and/or inner catheter reinforcement structure 50. In some embodiments, the inner catheter 26 may be considered a scaled-down version of the outer catheter 12, with the same elements but smaller dimensions.

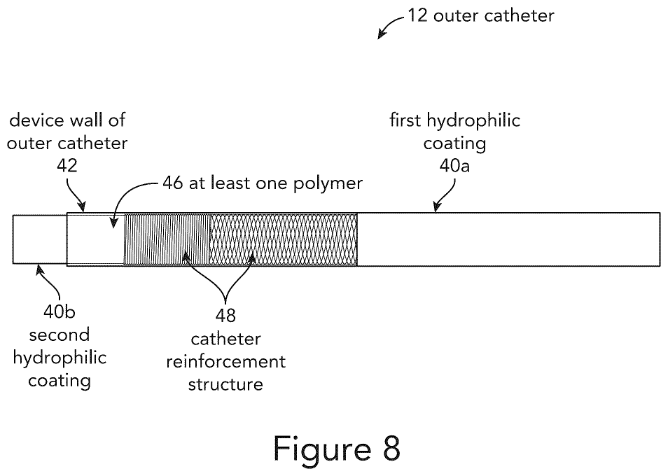

FIG. 8 is also similar to FIG. 6 in that it illustrates another view of the outer catheter 12, including the various layers of materials according to some embodiments. Included in FIG. 8 are the first hydrophilic coating 40a, the second hydrophilic coating 40b, the device wall 42, the at least one polymer 46, and the outer catheter reinforcement structure 48. In some embodiments, the device wall 42 may have a structure where the at least one polymer 46 and the outer catheter reinforcement structure 48 are melded together, with the first hydrophilic coating 40a located on the outer surface and the second hydrophilic coating 40b located on the inner surface.

In some embodiments, the device wall 42 has a “sandwich” structure comprising two layers of the at least one polymer 46 directly coupled together, with the outer catheter reinforcement structure 48 between the polymer layers 46. As previously discussed, the outer catheter reinforcement structure 48 may comprise a braid and/or coil structure. In some embodiments, the outer catheter reinforcement structure 48 and/or inner catheter reinforcement structure 50 may each include individual coil and/or braid structures, as indicated by the different appearances of the outer catheter reinforcement structure 48 in FIG. 8. For example, the coil structure is represented by the portion of the outer catheter reinforcement structure 48 to the left in FIG. 8, while the braid structure is represented by the portion of the outer catheter reinforcement structure 48 to the right in FIG. 8 according to some embodiments. In some embodiments, the “sandwich” style device wall 42 comprises an inner layer of at least one polymer 46, the coil structure on top of the inner polymer layer, the braid structure on top of the coil structure, and an outer layer of at least one polymer 46. In some embodiments, in the “sandwich” style, the device wall 42 also includes the first hydrophilic coating 40a and the second hydrophilic coating 40b.

In some embodiments, the “sandwich” style device wall 42 allows for a larger (more open) coil pitch in the coil structure, thereby enabling the outer catheter 12 to be softer than some embodiments where the coil structure has a tighter or more closed pitch. In some embodiments, a softer and more flexible outer catheter 12 can be desirable for certain uses, such as when navigating tortuous anatomy, to give the user (i.e., a medical practitioner) more freedom to move the device at different angles. In some embodiments, this “sandwich” style provides benefits from a manufacturing standpoint, as a more open coil pitch is easier to produce and may include a larger margin of error than a closed pitch. Some embodiments described herein are directed to a method of manufacture of the inner and/or outer catheters.

However, there are benefits to a device wall 42 comprising a tighter pitch coil structure. For example, in some embodiments, the outer catheter reinforcement structure 48 includes a coil defining a pitch smaller than 0.03 inches, and/or the second hydrophilic coating 40b is provided with a substantially solid and/or ribbed surface to adhere to. In this sense, the second hydrophilic coating 40b (as well as the fourth hydrophilic coating 40d of the inner catheter 26) may be thought of as having a textured, or “ribbed,” surface according to some embodiments. In comparison, in some embodiments, the first hydrophilic coating 40a (and the third hydrophilic coating 40c) may be thought of as having a substantially smooth surface. In some embodiments, the combination of textured and smooth surfaces of the hydrophilic coatings 40a-d provide just enough friction to allow a user to easily control movement of the outer catheter 12 and the inner catheter 26. For example, when the second hydrophilic coating 40b has a textured surface and the third hydrophilic coating 40c has a smooth surface, there may be enough friction between the two surfaces to prevent excessive and difficult-to-control sliding of the inner catheter 26 within the outer catheter 12, as may be the case if both hydrophilic coatings 40b, 40c were smooth.

In some embodiments, to ensure a sufficiently solid inner surface 20 of the outer catheter 12, the coil comprises a 0.002 inch round coil with a 0.004 inch pitch. In some embodiments, a tighter pitch coil may be better for facilitating lubricity of the inner surface 20 of the outer catheter 12. In some embodiments, a coil with a pitch less than 0.025 inches is desirable. In some embodiments, a sufficiently tight-pitch coil in the outer catheter reinforcement structure 48, combined with the second hydrophilic coating 40b on the inner surface 20 of the outer catheter 12, provides enough lubricity to replace the need for a liner, such as a PTFE liner, which is traditionally used in catheter construction. In some embodiments, the coil may comprise a round coil, a flat coil, and/or other types of coil design.

Regardless of the “style” of device wall 42 used (e.g., “sandwich” or tight-pitch coil), the use of a first and second hydrophilic coating 40a, 40b on the outer catheter 12 may allow for a thinner, more flexible device wall 42, as compared to other types of catheter walls without inner and outer coatings according to some embodiments. It should be noted that though FIG. 8 specifically labels the catheter as the outer catheter 12, the layers shown in FIG. 8 and the preceding discussion also apply to the inner catheter 26 in some embodiments, such that FIG. 8 may be considered as depicting either the outer catheter 12 or the inner catheter 26.

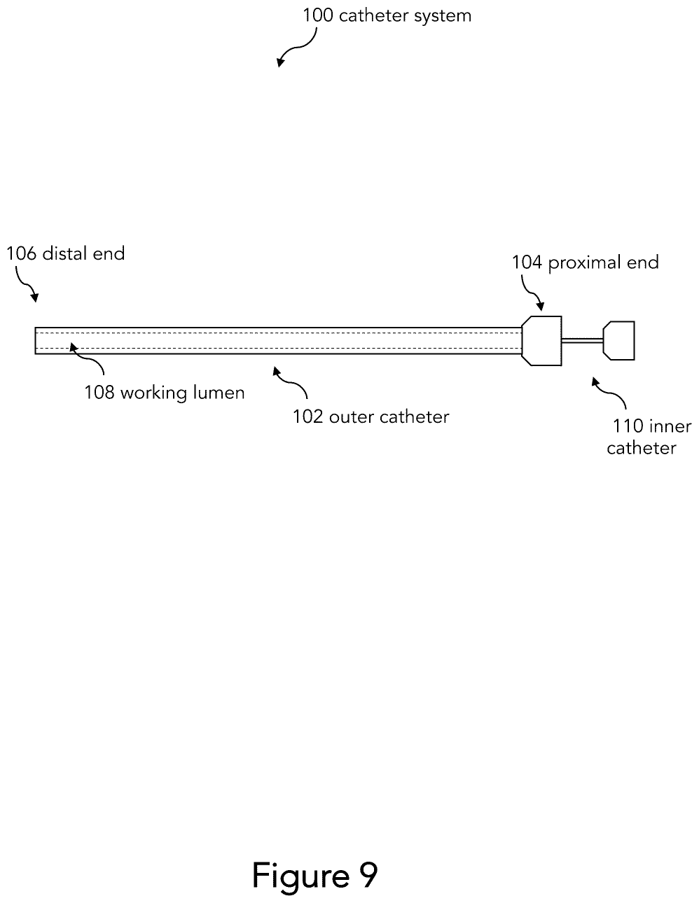

FIG. 9 illustrates a catheter system 100 comprising an outer catheter 102 and an inner catheter 110, which may be substantially similar in construction to outer catheter 12 and/or inner catheter 26 according to some embodiments. As shown, the outer catheter 102 may include a proximal end 104 and a distal end 106 located opposite the proximal end 104. In some embodiments, the outer catheter 102 includes a working lumen 108 extending between the proximal end 104 and the distal end 106. In some embodiments, the working lumen 108 includes the space inside the catheter 110 configured to allow the passage of various instruments, such as guidewires, microcatheters, stent retrievers, and/or is used for the aspiration of clots and other obstructions within a patient's vasculature. In some embodiments, the working lumen 108 is configured to at least partially receive the inner catheter 110.

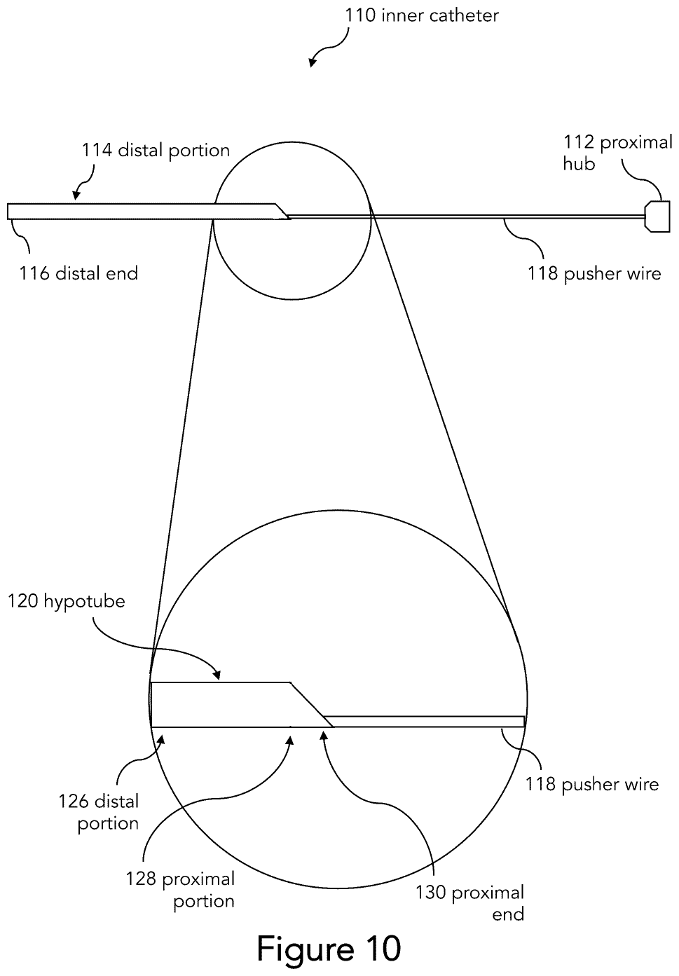

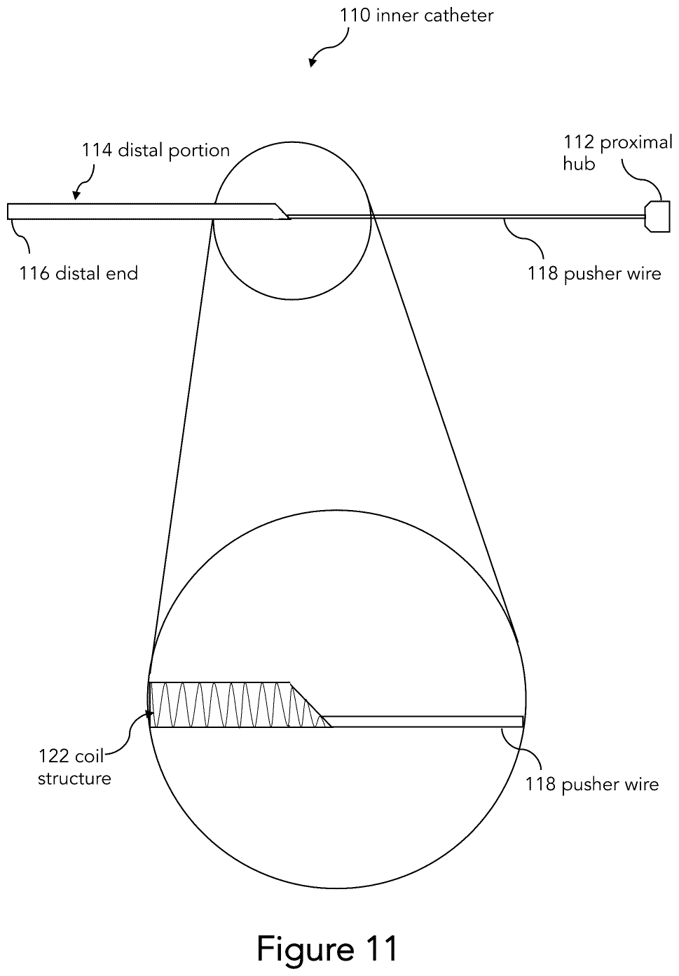

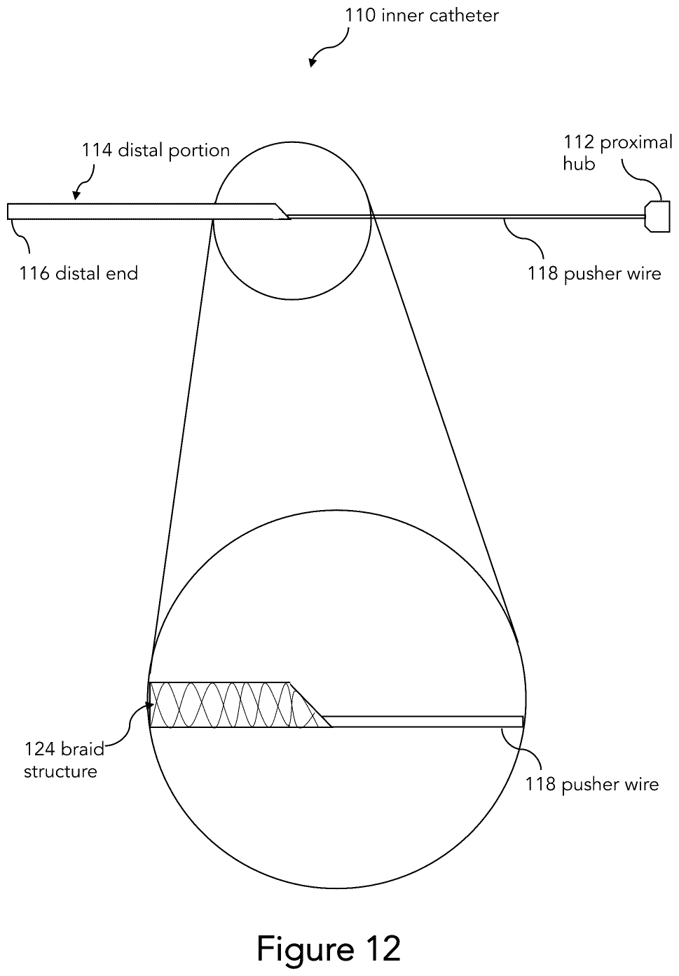

FIGS. 10-12 illustrate the inner catheter 110, which, in some embodiments, comprises a proximal hub 112, a distal portion 114 having a distal end 116 located opposite the proximal hub 112, and a pusher wire 118 extending between the proximal hub 112 and the distal portion 114. In some embodiments, the working lumen 108 is configured to at least partially receive the pusher wire 118, as shown in FIG. 9. As illustrated in the inset view of FIG. 10, in some embodiments, the distal portion 114 may comprise a hypotube 120. In some embodiments, the hypotube 120 comprises a stainless steel hypotube. In some embodiments, the hypotube 120 may comprise a nitinol hypotube. In some embodiments, the hypotube 120 is comprised of any suitable material, and, in some embodiments, is a laser-cut hypotube. In some embodiments, the hypotube 120 may comprise a non-laser-cut hypotube. In some embodiments, the hypotube 120 comprises a distal portion 126 and a proximal portion 128 located opposite the distal portion 126. As shown, the proximal portion 128 is configured to taper to a proximal end 130 coupled to the pusher wire 118. Rather than a hypotube 120, the distal portion 114 of the inner catheter 110 may comprise a coil structure 122, as illustrated in FIG. 11, and/or a braid structure 124, as illustrated in FIG. 12 according to some embodiments. In some embodiments, the distal portion 114 of the inner catheter 110 comprises a combination of the coil structure 122 and the braid structure 124. In some embodiments, the distal portion 114 may comprise any suitable material configuration and is not limited to the examples shown in the Figures and discussed in this disclosure.

As shown in FIGS. 10-12, and due to the inclusion of the pusher wire 118, in some embodiments, the distal portion 114 of the inner catheter 110 includes a length substantially less than the full length of the inner catheter 110. In comparison, the outer catheter 102 shown in FIG. 9 may comprise a single tube defining substantially the full length of the outer catheter 102, minus the proximal end 104 according to some embodiments. In some embodiments, in the catheter system 100, the outer catheter 102 may be considered a “full catheter” and the inner catheter 110 may be considered a “partial catheter.” In some embodiments, the distal portion 114 of the inner catheter 110, whether a hypotube 120, coil structure 122, and/or braid structure 124, may include a length of about twenty centimeters. In some embodiments, the distal portion 114 defines a length less than twenty centimeters. In some embodiments, the distal portion 114 may define a length greater than twenty centimeters.

In some embodiments, the pusher wire 118 is fixedly coupled (e.g., via welding, bonding, adhesive, or the like) to the distal portion 114 of the inner catheter 110. In some embodiments, the pusher wire 118 is configured to facilitate navigation of the inner catheter 110 through the working lumen 108 of the outer catheter 102. For example, according to a method of use, during a procedure, a physician (or another qualified medical professional) is configured to “push” the inner catheter 110 through the outer catheter 102 using the proximal hub 112 and/or the pusher wire 118 according to some embodiments. In some embodiments, the relative rigidity of the pusher wire 118 may help advance the inner catheter 110 with limited twisting, kinking, bending, etc. of the distal portion 114. In some embodiments, the pusher wire 118 comprises a round wire. In some embodiments, the pusher wire 118 may comprise a flat wire.

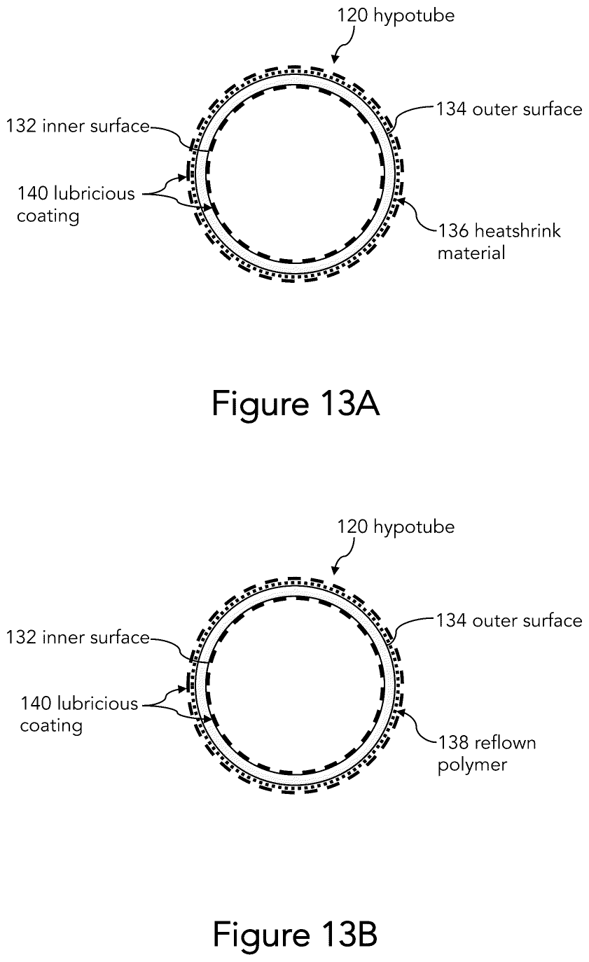

Turning now to FIGS. 13A and 13B, cross-sectional views of the hypotube 120 according to some embodiments are shown. In some embodiments, the hypotube 120 comprises an inner surface 132 and an outer surface 134 located opposite the inner surface 132. In some embodiments, the outer surface 134 is covered in a heatshrink material 136, as shown in FIG. 13A. In some embodiments, the heatshrink material 136 comprises a material laminated, fused, and/or melted onto the outer surface 134 of the hypotube 120. In some embodiments, at least a portion of the heatshrink material 136 and at least a portion of the inner surface 132 of the hypotube 120 are coated with a lubricious coating 140. In some embodiments, substantially the entirety of the heatshrink material 136 and substantially the entirety of the inner surface 132 are coated with the lubricious coating 140. In some embodiments, at least a portion of the heatshrink material 136 and substantially the entirety of the inner surface 132 is coated with the lubricious coating 140. In some embodiments, substantially the entirety of the heatshrink material 136 and at least a portion of the inner surface 132 is coated with the lubricious coating 140. In some embodiments, the inner surface 132 varies in diameter along the length of the catheter.

In some embodiments, the lubricious coating 140 may comprise a hydrophilic coating. In some embodiments, the lubricious coating 140 comprises silicone. In some embodiments, the lubricious coating 140 may comprise any suitable type of coating, and is not intended to be limited to the examples discussed in this disclosure. In some embodiments, the lubricious coating 140 helps facilitate smooth navigation of the hypotube 120 through the working lumen 108 of the outer catheter 102. In some embodiments, where the inner catheter 110 extends distally from the outer catheter 102, the lubricious coating 140 may also help facilitate smooth navigation of the hypotube 120 through a patient's vasculature. In some embodiments, the lubricious coating 140 on the inner surface 132 of the hypotube 120 facilitates smooth movement of a secondary device (e.g., a guidewire, microcatheter, specialized device, etc.) through the hypotube 120.

FIG. 13B illustrates that, in some embodiments, the outer surface 134 of the hypotube 120 is covered in a reflown polymer 138 rather than a heatshrink material 136 according to some embodiments. In some embodiments, at least a portion of the reflown polymer 138 and at least a portion of the inner surface 132 of the hypotube 120 is coated with the lubricious coating 140. In some embodiments, substantially the entirety of the reflown polymer 138 and substantially the entirety of the inner surface 132 of the hypotube 120 are coated with the lubricious coating 140. In some embodiments, at least a portion of the reflown polymer 138 and substantially the entirety of the inner surface 132 is coated with the lubricious coating 140. In some embodiments, substantially the entirety of the reflown polymer 138 and at least a portion of the inner surface 132 is coated with the lubricious coating 140.

In some embodiments, the outer surface 134 of the hypotube 120 is covered with a combination of the heatshrink material 136 and the reflown polymer 138. In some embodiments, at least a portion of the hypotube 120 includes a PTFE liner rather than the lubricious coating 140. For example, in some embodiments, half of the hypotube 120 may include a PTFE liner while the other half includes the lubricious coating 140. In some embodiments, half of the hypotube 120 may include a PTFE liner while the other half includes no lubricious coating 140. In some embodiments, the hypotube 120 may also include neither a PTFE liner nor a lubricious coating 140. In some embodiments, the catheter system 100 including the coil structure 122 and/or braid structure 124, as illustrated in FIGS. 11 and 12, respectively, may also include a heatshrink material 136, reflown polymer 138, or combination thereof to cover the coil structure 122 and/or braid structure 124, as applicable. In some embodiments, the coil structure 122 and/or braid structure 124 may include the lubricious coating 140 as illustrated in FIGS. 13A and 13B.

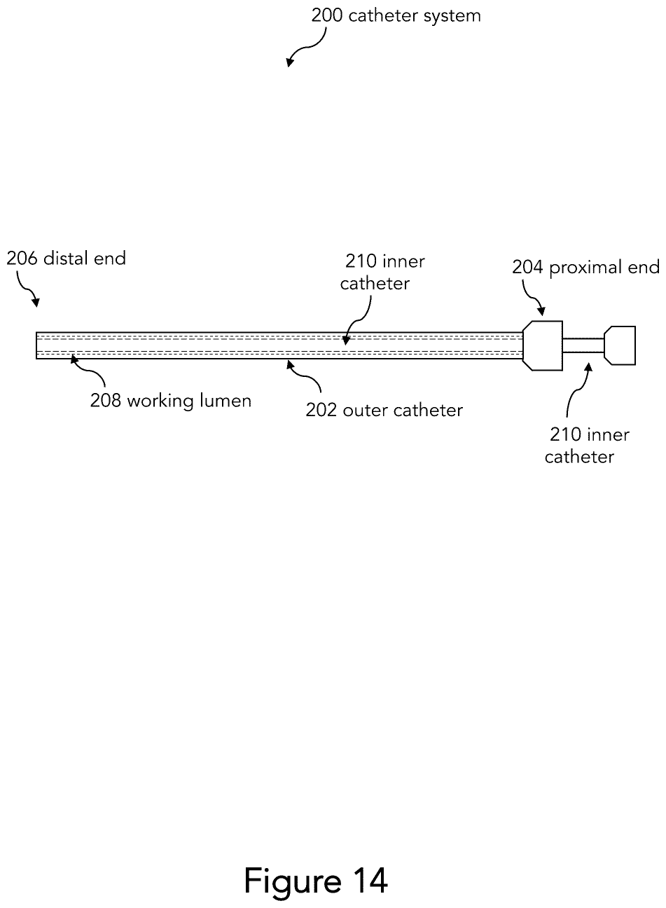

Referring now to FIG. 14, a catheter system 200 is shown according to some embodiments. In some embodiments, the catheter system 200 comprises an outer catheter 202 having a proximal end 204, a distal end 206 located opposite the proximal end 204, and a working lumen 208 extending between the proximal end 204 and the distal end 206. In some embodiments, the catheter system 200 may also include an inner catheter 210, and the working lumen 208 is configured to at least partially receive the inner catheter 210, as demonstrated in FIG. 14. As with any catheter described herein according to some embodiments, the outer catheter 202 and inner catheter 210 may be made by similar manufacturing methods and/or comprise similar structures, such as those described in relation to FIGS. 6-8 and 23.

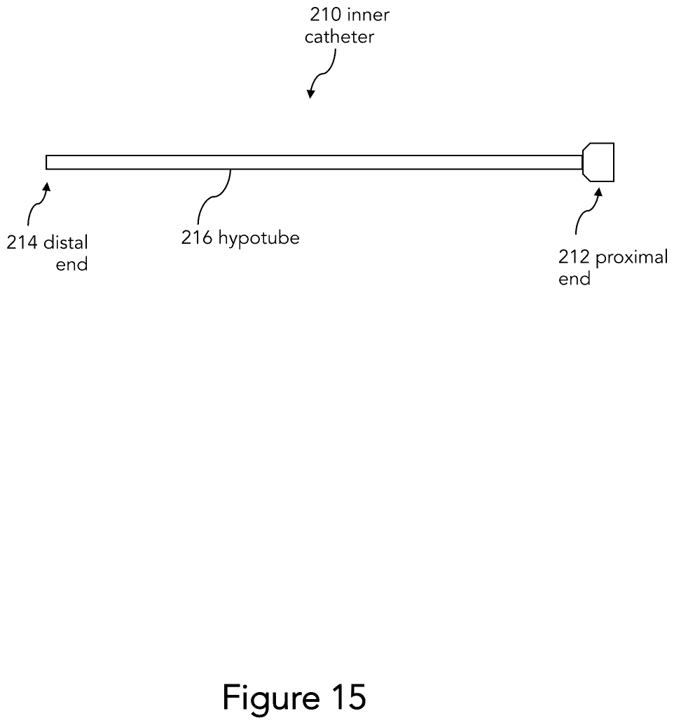

FIG. 15 illustrates the inner catheter 210 in more detail, including the proximal end 212 and the distal end 214 located opposite the proximal end 212. Unlike the inner catheter 110 of the catheter system 100 (shown in FIGS. 9-13) according to some embodiments, the inner catheter 210 may comprise a “full” catheter rather than a “partial” catheter. Stated differently, in some embodiments, the inner catheter 210 may comprise a hypotube 216 configured to extend the full length from the proximal end 212 to the distal end 214. Similar to the hypotube 120 of the inner catheter 110, in some embodiments, the hypotube 216 of the inner catheter 210 may comprise a laser-cut hypotube. In some embodiments, the inner catheter 210 may comprise a non-laser-cut hypotube. In some embodiments, the hypotube 216 comprises a stainless steel hypotube. In some embodiments, the hypotube 216 may comprise a nitinol hypotube, or a hypotube constructed of any other suitable material.

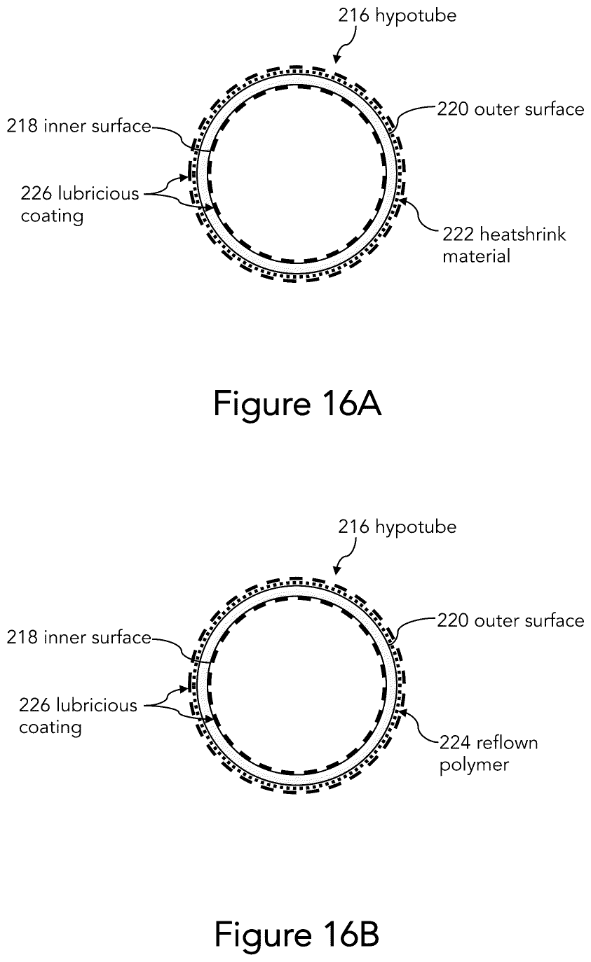

FIGS. 16A and 16B are similar to FIGS. 13A and 13B, though they illustrate the hypotube 216 rather than the hypotube 120 according to some embodiments. FIGS. 16A and 16B show cross-sectional views of the hypotube 216, wherein, in some embodiments, the hypotube 216 comprises an inner surface 218 and an outer surface 220 located opposite the inner surface 218. In some embodiments, the outer surface 220 is covered in a heatshrink material 222, as shown in FIG. 16A. In some embodiments, the heatshrink material 222 may comprise a material laminated or melted onto the outer surface 220 of the hypotube 216. In some embodiments, at least a portion of the heatshrink material 222 and at least a portion of the inner surface 218 of the hypotube 216 are coated with a lubricious coating 226. In some embodiments, substantially the entirety of the heatshrink material 222 and substantially the entirety of the inner surface 218 are coated with the lubricious coating 226. In some embodiments, at least a portion of the heatshrink material 222 and substantially the entirety of the inner surface 218 is coated with the lubricious coating 226. In some embodiments, substantially the entirety of the heatshrink material 222 and at least a portion of the inner surface 218 is coated with the lubricious coating 226.

In some embodiments, the lubricious coating 226 is substantially similar to the lubricious coating 140 of the catheter system 100. In some embodiments, the lubricious coating 226 may comprise a hydrophilic coating. In some embodiments, the lubricious coating 226 comprises silicone. In some embodiments, the lubricious coating 226 may comprise any suitable type of coating, and is not intended to be limited to the examples discussed in this disclosure. In some embodiments, the lubricious coating 226 helps facilitate smooth navigation of the hypotube 216 through the working lumen 208 of the outer catheter 202. In some embodiments, where the inner catheter 210 extends distally from the outer catheter 202, the lubricious coating 226 may also help facilitate smooth navigation of the hypotube 216 through a patient's vasculature. In some embodiments, the lubricious coating 226 on the inner surface 218 of the hypotube 216 facilitates smooth movement of a secondary device (e.g., a guidewire, microcatheter, specialized device, etc.) through the hypotube 216, similar to some embodiments described herein.

FIG. 16B illustrates that, in some embodiments, the outer surface 220 of the hypotube 216 is covered in a reflown polymer 224 rather than a heatshrink material 222. In some embodiments, at least a portion of the reflown polymer 224 and at least a portion of the inner surface 218 of the hypotube 216 is coated with the lubricious coating 226. In some embodiments, substantially the entirety of the reflown polymer 224 and substantially the entirety of the inner surface 218 of the hypotube 216 are coated with the lubricious coating 226. In some embodiments, at least a portion of the reflown polymer 224 and substantially the entirety of the inner surface 218 is coated with the lubricious coating 226. In some embodiments, substantially the entirety of the reflown polymer 224 and at least a portion of the inner surface 218 is coated with the lubricious coating 226.

In some embodiments, the outer surface 220 of the hypotube 216 is covered with a combination of the heatshrink material 222 and the reflown polymer 224. In some embodiments, at least a portion of the hypotube 216 includes a PTFE liner rather than the lubricious coating 226. For example, in some embodiments, half of the hypotube 216 may include a PTFE liner while the other half includes the lubricious coating 226. In some embodiments, half of the hypotube 216 may include a PTFE liner while the other half includes no lubricious coating 226. In some embodiments, the hypotube 216 may also include neither a PTFE liner nor a lubricious coating 226. In some embodiments, the inner catheter 210 may comprise, rather than the hypotube 216, a coil structure and/or braid structure, similar to those illustrated in FIGS. 6, 7, 8, 11 and 12. In some embodiments, the inner catheter 210 comprising a coil and/or braid structure also includes a heatshrink material 222, reflown polymer 224, or combination thereof to cover the coil structure and/or braid structure, as applicable. In addition, embodiments with the coil structure and/or braid structure may include the lubricious coating 226 as illustrated in FIGS. 16A and 16B.

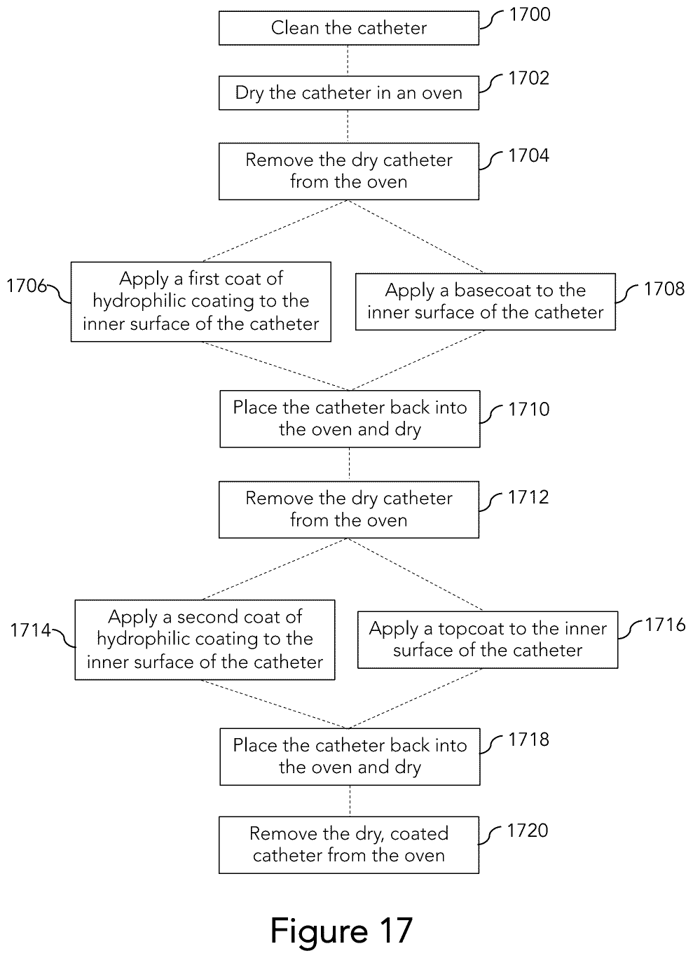

FIG. 17 shows a flowchart illustrating a nonlimiting example process of coating and curing an inner surface of a catheter according to some embodiments. For the purposes of this disclosure, the “catheter” recited in FIG. 17 may comprise elements of the catheter system 10 (i.e., the outer catheter 12 or the inner catheter 26), elements of the catheter system 100 (i.e., the outer catheter 102 or the inner catheter 110), and/or elements of the catheter system 200 (i.e., the outer catheter 202 or the inner catheter 210). The steps of the process, which include a method of manufacture, should be considered as applying to any of the catheters recited in this disclosure.

In some embodiments, the process shown in FIG. 17 starts with cleaning the catheter, at step 1700. In some embodiments, cleaning the catheter includes flushing the catheter with purified water, isopropyl alcohol (“IPA”), a mix of IPA and water, and/or some other suitable cleansing fluid. In some embodiments, the next step is to dry the catheter in an oven, at step 1702. In some embodiments, the drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 1704: remove the dry catheter from the oven.

Next, in some embodiments, the process can continue in one of two possible steps. One option is to apply a first coat of hydrophilic coating to the inner surface of the catheter, shown at step 1706 according to some embodiments. In some embodiments, a basecoat is applied to the inner surface of the catheter, at step 1708. Both steps 1706 and 1708 may use positive or negative pressure to fill the catheter with either the hydrophilic coating (step 1706) or the basecoat (step 1708) according to some embodiments. In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

After either step 1706 or step 1708, in some embodiments the process may continue to place the catheter back into the oven to dry, at step 1710. Similar to the first drying step (i.e., step 1702), in some embodiments step 1710 may involve placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. Step 1710 is considered a “heat curing” step, as heat is used to dry (i.e., cure) the coating according to some embodiments. Next, in some embodiments, the positive or negative pressure source is disconnected and the dry catheter is removed from the oven, at step 1712.

At this point, in some embodiments, the process again diverges into two different options. In some embodiments, one option is to apply a second coat of hydrophilic coating to the inner surface of the catheter, at step 1714. In some embodiments, the other option is to apply a topcoat to the inner surface of the catheter, at step 1716. Similar to the application of the first coat of hydrophilic coating (at step 1706) and the application of the basecoat (at step 1708), in some embodiments, both steps 1714 and 1716 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with placing the catheter back into the oven to dry (or “heat cure”) again, at step 1718. Like the first and second drying steps (step 1702 and step 1710), in some embodiments, step 1718 may involve placing the catheter in an oven set to a temperature between 0° C. and 400° C. and/or applying positive or negative pressured air (e.g., oxygen, a mix of oxygen and nitrogen, etc.) to the hub of the catheter in order to dry the inner surface of the catheter. In some embodiments, the process concludes by disconnecting the positive or negative pressure source and removing the dry, coated catheter from the oven, at step 1720.

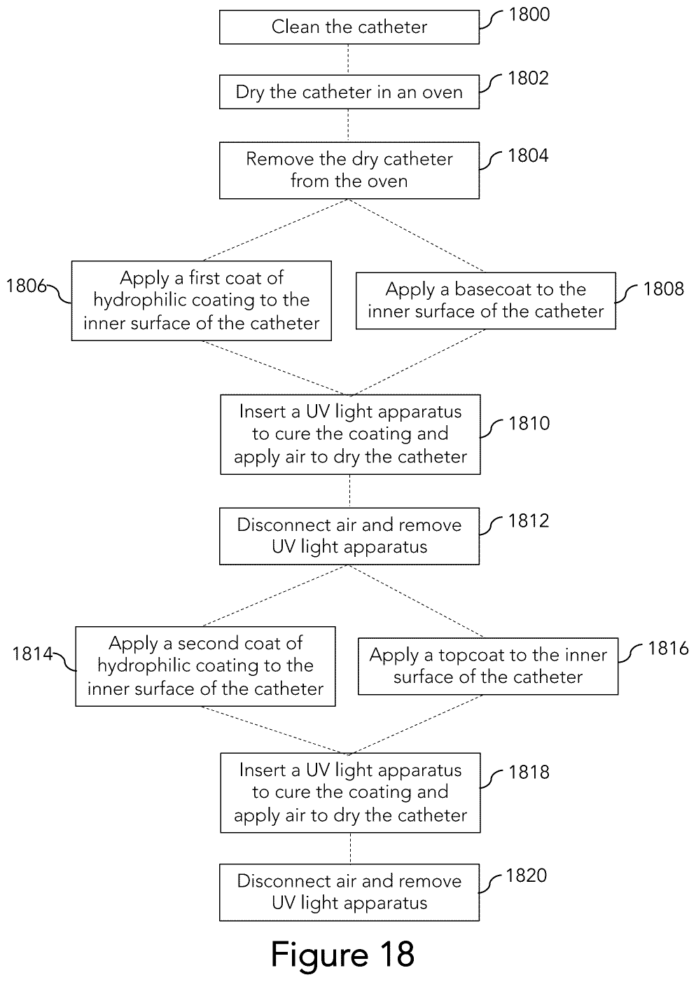

FIG. 18 is similar to FIG. 17, and includes a flowchart illustrating a slightly different nonlimiting example process of coating and curing an inner surface of a catheter according to some embodiments. As with the process shown in FIG. 17, for the purposes of this disclosure, the “catheter” recited in FIG. 18 may comprise elements of the catheter system 10 (i.e., the outer catheter 12 or the inner catheter 26), elements of the catheter system 100 (i.e., the outer catheter 102 or the inner catheter 110), elements of the catheter system 200 (i.e., the outer catheter 202 or the inner catheter 210), and/or catheter 300. The steps of the process should be considered as applying to any of the catheters recited in this disclosure according to some embodiments.

In some embodiments, the process shown in FIG. 18 starts with cleaning the catheter, at step 1800. In some embodiments, cleaning the catheter includes flushing the catheter with purified water, IPA, a mix of IPA and water, or some other suitable cleansing fluid. In some embodiments, the next step is to dry the catheter in an oven, at step 1802. The drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 1804: removing the dry catheter from the oven.

Next, in some embodiments, the process can continue in one of two possible steps. In some embodiments, one option is to apply a first coat of hydrophilic coating to the inner surface of the catheter, shown at step 1806. In some embodiments, a basecoat is applied to the inner surface of the catheter, at step 1808. In some embodiments, both steps 1806 and 1808 may use positive or negative pressure to fill the catheter with either the hydrophilic coating (step 1806) or the basecoat (step 1808). In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time. In some embodiments, a reduction in flow is used to increase the dwell time within a catheter.

After either step 1806 or step 1808, in some embodiments, the process may continue by inserting a UV light apparatus to cure the coating and applying positive or negative pressured air (e.g., oxygen, a mix of oxygen and nitrogen, etc.) to the hub of the catheter in order to dry the inner surface of the catheter, at step 1810. In some embodiments, the UV light apparatus is inserted into the inner diameter of the catheter to cure the coating on the inner surface. Next, the positive or negative pressure source is disconnected and the UV light apparatus is removed from the catheter, at step 1812, according to some embodiments.

At this point, in some embodiments, the process again diverges into two different options. In some embodiments, one options is to apply a second coat of hydrophilic coating to the inner surface of the catheter, at step 1814. In some embodiments, the other option is to apply a topcoat to the inner surface of the catheter, at step 1816. Similar to the application of the first coat of hydrophilic coating (at step 1806) and the application of the basecoat (at step 1808), in some embodiments, both steps 1814 and 1816 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with another round of UV light curing, at step 1818. Like the first UV curing step (step 1810), step 1818 may involve inserting a UV light apparatus to cure the coating and applying positive or negative pressured air (e.g., oxygen, a mix of oxygen and nitrogen, etc.) to the hub of the catheter in order to dry the inner surface of the catheter. In some embodiments, the UV light apparatus is inserted into the inner diameter of the catheter to cure the coating on the inner surface. In some embodiments, the process includes disconnecting the positive or negative pressure source and removing the UV light apparatus from the catheter, at step 1820.

The catheter system 10 is configured for use in various procedures conducted in a variety of locations of a patient's anatomy. Though brain-specific thrombectomy is discussed, the disclosure should not be considered limiting to any specific type or location of the procedure. The catheter system 10 is used for the aspiration of clots throughout a patient's body, and the various aspects of the catheter system 10 discussed above may improve the rate of clot removal in a number of procedure locations.

Catheter systems may include a full outer catheter 102 and partial inner catheter 110, like the catheter system 100, or may include a full outer catheter 202 and a full inner catheter 210, like the catheter system 200. In some embodiments, a catheter system includes a partial outer catheter and a full inner catheter. In some embodiments, a catheter system may also include a partial outer catheter and a partial inner catheter.

Though not shown in the figures, a method of using any of the catheter systems described herein according to some embodiments, such as the catheter system 10, the catheter system 100, the catheter system 200, and/or catheter shaft 300 comprises inserting an outer catheter, such as the outer catheter 12, the outer catheter 102, and/or the outer catheter 202, into a patient's vasculature, wherein the outer catheter includes a proximal end and a distal end located opposite the proximal end, advancing the outer catheter through the patient's vasculature toward a vascular lesion, and advancing the outer catheter to a location selected from the group consisting of a first location and a second location. In some embodiments, the first location is within a first predetermined distance from the vascular lesion, and the second location is within a second predetermined distance from the vascular lesion. In some embodiments, when the outer catheter is in the first location, the outer catheter is able to aspirate the vascular lesion, and when the outer catheter is in the second location, the outer catheter is unable to aspirate the vascular lesion. In some embodiments, when the outer catheter is in the first location, the method further comprises aspirating the vascular lesion with the outer catheter. In some embodiments, when the outer catheter is in the second location, the method may further comprise advancing an inner catheter, such as the inner catheter 26, the inner catheter 110, and/or the inner catheter 210, through the outer catheter toward the first location. In some embodiments, when the inner catheter is in the first location, the method further comprises aspirating the vascular lesion with the inner catheter.

In some embodiments, a fluoropolymer-free manufacturing process for the catheter begins by selecting a PTFE mandrel that matches the desired inner diameter of the catheter. In some embodiments, one or more reinforcement structures are placed on and/or over the mandrel. In some embodiments, a reinforcement structure includes a wire, string, coil, and/or laser cut hypotube. In some embodiments, different reinforcement structures are placed in different regions of the mandrel and/or are overlaid on each other. In some embodiments, the reinforcement structure includes one or more of a flat surface, a round surface, or some combination of the two.

In some embodiments, the reinforcement structure includes a coil structure, which may include elements such as one or more flat wires and/or one or more round wires as discussed above. While some embodiments may describe the use of one element type (e.g., wire, string) or element shape (e.g., round, flat), it is understood that the specific elements and/or element shapes are interchangeable when describing the metes and bounds of the system, and reference to an “element” is a reference to any combination of element types and/or shapes described herein.

In some embodiments, to form the coil structure, a wire is wound over the polytetrafluoroethylene (PTFE) mandrel using a chosen element, shape, and/or pitch, after which the coil structure is terminated. In some embodiments, the coil structure is configured to leave recesses in the inner diameter. In some embodiments, the coil structure is configured to impart a rib pattern comprising peaks and valleys on the inner diameter of the catheter.

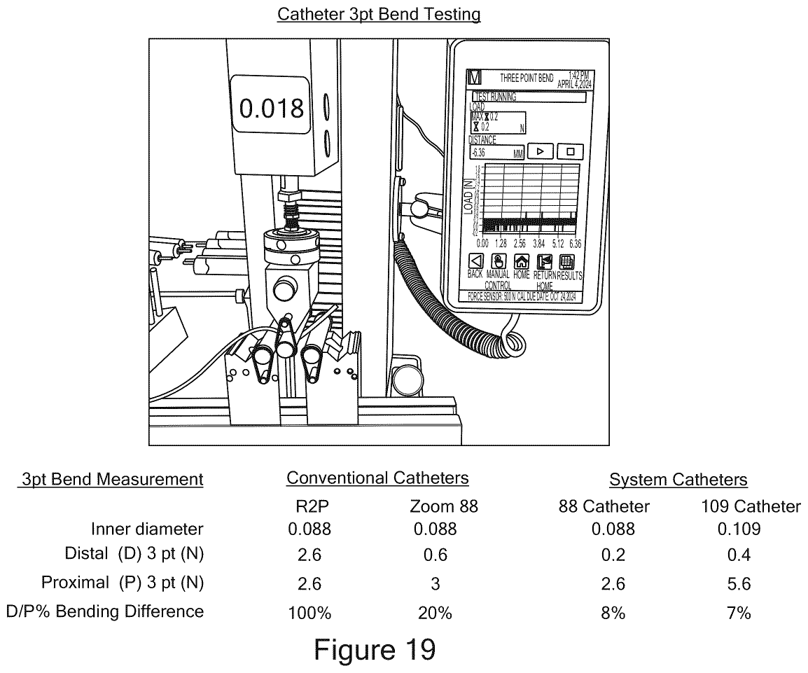

In some embodiments, a braid is then applied over the coiled PTFE mandrel, utilizing the selected material, braid pattern, and braid picks per inch (PPI) to obtain a desired structural integrity. However, it has been found that the coil structure alone, in accordance with some embodiments, provides sufficient properties to achieve the results shown in FIGS. 19-22.

In some embodiments, the reinforcement structure is wound around the mandrel in a braided pattern. In some embodiments, the braided pattern is configured to leave recesses in the inner diameter. A non-limiting recess shape includes a polygon shape, which may include a diamond or square pattern and/or grid pattern according to some embodiments. Braiding patterns formed using Steeger USA® machines have been found to produce acceptable results and provide a variety of patterns including flat braids, square braids, spiral braids, strands, and coils.

In some embodiments, the recess shape for any reinforcement structure described herein is configured to create peaks and valleys along the inner diameter of the catheter, where the valleys are configured to reduce the contact area of the inner diameter by 15-85%. In some embodiments, the peaks define the inner diameter contact area for a substantially smooth portion of a traversing structure (e.g., guide wire). In some embodiments, the peaks include a flat surface, such as in the case of a flat wire coil structure and/or hypotubes. Flat wires may increase stiffness and/or compressive strength (including vacuum strength) but may have greater contact area against a surface of a traversing structure according to some embodiments. Round wires may decrease stiffness and/or compressive strength but generate less friction for a traversing structure due to a smaller surface area at a round peak as compared to a flat peak.

In some embodiments, the reinforcement structure includes one or more hypotubes. In some embodiments, a hypotube may include one or more hollow portions. In some embodiments, the hollow portions include material removed from the hypotube in a pattern shape. In some embodiments, a step includes creating a pattern shape in the form of one or more holes longitudinal slots, spiral (helical) cuts, circular (ring) cuts, intersecting grids (e.g., mesh, crisscross lines), and/or any custom geometric pattern. In some embodiments, pattern shapes are configured to impart specific functionality such as expansion, flexibility, or kink resistance.

In some embodiments, the use of laser cutting enables the creation of precise and intricate patterns along the hypotube. Laser cutting provides clean cuts with minimal burrs and heat-affected zones, ensuring the structural integrity and smoothness of the tube in accordance with some embodiments. In some embodiments, the hypotubes is made from biocompatible metals, such as stainless steel or nickel-titanium alloys (Nitinol), which offer excellent strength and flexibility, as well as resistance to corrosion.

In some embodiments, the reinforcement structure forming step, a platinum iridium marker band is positioned over the braid. In some embodiments, the ends of the braid are trimmed flush with the marker band, and the braid is bonded to the marker band using a urethane-based adhesive. This ensures the marker band is securely attached and the transition between materials is smooth in some embodiments.

In some embodiments, a next phase includes loading each of the polymer extrusions, or tubes, over the coiled and braided reinforcement structure while on the mandrel. In some embodiments, this process starts with the stiffest polymer (e.g., ML24) and concludes with the softest (e.g., 42 A Neusoft), which is placed adjacent to the marker band. This step, in some embodiments, creates a gradient of flexibility along the length of the catheter, providing both reinforcement and pliability where needed.

In some embodiments, an expanded fluorinated ethylene propylene (FEP) heat shrink is then loaded over the polymer extrusions, and the assembly is placed into a reflow machine. In some embodiments, the reflow machine employs heated forced air to melt the polymer extrusions, causing them to fuse together and bond to the metallic reinforcement structure. During this process, at least a portion of the polymer flows through the hollow portions of the reinforcement structure. In some embodiments, at least a portion of the polymer coats an inner diameter of the reinforcement structure, sealing the catheter. In some embodiments, at least a portion of the inner diameter is not coated by the polymer, and/or at least a peak surface of the inner diameter is not coated with a polymer. In some embodiments, a peak surface of the inner diameter is coated with a polymer. In some embodiments, polymer located in the area of a hollow portion creates a valley on the inner diameter of the catheter.

After the reflow process, the FEP heat shrink is removed, and the PTFE mandrel is extracted from the assembly according to some embodiments. In some embodiments, the formed catheter does not include PTFE and/or a PTFE liner along the inner diameter. Some embodiments include a step to over mold a hub onto the stiff (e.g., ML24) end of the tube, creating a secure connection point for the catheter. In some embodiments, the hub is attached to the catheter before any hydrophilic coating is applied to the catheter as described above. In some embodiments, an extended portion of polymer material is left in front of the marker band to facilitate subsequent hydrophilic coating processing, which will enhance the catheter's lubricity and ease of use during medical procedures.

In some embodiments, the catheter is then prepared for the hydrophilic coating processing by cleaning the outer diameter, which may include using a wipe saturated with a cleaning agent (e.g., 70% isopropyl alcohol (IPA)) to ensure a clean surface for coating adherence. Some embodiments include a step of coupling a fluid source (e.g., 20 cc syringe) to the catheter at the hub and flushing the catheter interior with a cleaning agent (e.g., 70% IPA) to remove any contaminants that may interfere with the coating process.

Following the flush, a hydrophilic basecoat is aspirated into the catheter using a vacuum pump (e.g., another 20 cc syringe) to coat the inner diameter of the catheter. To coat the outer diameter, the catheter is at least partially submerged into a container filled with hydrophilic fluid to a specific depth, which is carefully controlled to achieve the desired coating thickness according to some embodiments. Once the desired coating depth is reached, the vacuum is released, enabling the hydrophilic fluid to drain from the inner diameter in some embodiments. In some embodiments, the catheter is withdrawn from the hydrophilic fluid at a consistent rate to ensure an even coating. In some embodiments, the rate of withdrawal affects surface tension which determines coating thickness.

The catheter is then placed in an oven set to a curing temperature of 50-70° C. (e.g., 60° C.) for a duration of 20-40 (e.g., 30) minutes according to some embodiments. In some embodiments, an airline is connected to the hub during this time, which facilitates the curing of the hydrophilic basecoat on both the inner and outer diameters simultaneously. In some embodiments, air is slowly forced through the catheter. By adjusting the rate of air flow, in some embodiments, it is possible to control the thickness of the hydrophilic coating on the inner diameter. A slower air flow allows more time for the coating to set, potentially leading to a thicker coating, while a faster air flow thins out the coating and/or speeds up the drying process according to some embodiments.

In some embodiments, the process is repeated for a hydrophilic topcoat. In some embodiments, a 20 cc syringe is used to aspirate the topcoat into the catheter, which is then dipped into a container of hydrophilic top coat to the predetermined depth to achieve the desired coating thickness. After reaching the desired depth, in some embodiments the catheter is flushed (e.g., by releasing the vacuum and/or syringe) to remove any excess topcoat and then removed steadily.

In some embodiments, the catheter is placed back into the oven at a curing temperature of 50-70° C. (e.g., 60° C.) for a duration of 20-40 (e.g., 30) minutes. In some embodiments, the airline is still connected to the hub and/or the airline is connected to the hub. In some embodiments, air is supplied to the inner diameter at a same or similar temperature as the curing temperature, allowing the hydrophilic topcoat to cure on both the inner and outer diameters of the catheter simultaneously. In some embodiments, the sacrificial extended tip of the catheter is cut off, and the tip is rounded to ensure a smooth and/or finished end ready for medical use.

In some embodiments, the Fluoropolymer Free Manufacturing process includes constructing a catheter system that is compatible with hydrophilic coatings. In some embodiments, the catheter structure and/or manufacturing process does not include the use of fluoropolymers within the catheter and instead utilizes a range of materials that are conducive to the application of hydrophilic coatings.

In some embodiments, the catheter's reinforcement structure may include a stainless steel coil, with alternative material options such as nitinol or tungsten. As discussed previously, the reinforcement structure (e.g., coil structure) cross-section can be either round or flat in shape according to some embodiments. Similarly, the catheter may include a stainless steel braid for additional reinforcement in some embodiments, with the same alternative material options and the choice between round and/or flat types of braid. In some embodiments, as an alternative and/or in addition to using coil and/or braid components, the construction may employ a laser-cut hypotube made from Stainless Steel or Nitinol. Depending on application requirements, the catheter may feature just a coil, just a braid, or just a hypotube for reinforcement.

In some embodiments, the termination of the reinforcement structure is achieved using a urethane-based UV adhesive, ensuring a secure end. In some embodiments, the catheter's tubing transitions from rigid to soft materials, starting with Nylon tubing, specifically Vestimid ML24, which is the most rigid polymer used in catheters, followed by Vestimid ML21, the second most rigid polymer, according to some embodiments.

To create a smooth transition in flexibility, in some embodiments, PEBAX tubing is used in the middle of the device. The PEBAX material comes in varying hardness levels, denoted by the “D” hardness scale, with higher numbers indicating greater rigidity. In some embodiments, the catheter utilizes PEBAX tubing in descending order of rigidity, from 72 D to 25 D.

For the tip of the device, which requires maximum navigability, Urethane tubing is employed in some embodiments. The “D” hardness scale intersects with the “A” hardness scale at 25 D, approximately equivalent to 80 A. In some embodiments, the Urethane tubing used progresses from 25 D at the proximal end (i.e., adjacent to the hub) to the softer 42 A Neusoft as the distal end. In some embodiments, at the very tip (distal end) of the catheter, a Platinum Iridium marker band is incorporated. This marker band allows physicians to visually confirm the catheter's tip location under fluoroscopy according to some embodiments.

In some embodiments, the catheter's female hub connector is constructed by overmolding 72 D PEBAX onto the Nylon end of the device. Alternatively, a pre-molded hub is attached using UV adhesive in some embodiments. To ensure a smooth transition from the Nylon shaft to the 72 D PEBAX hub, Polyolefin heat shrink is used as a strain relief in some embodiments, providing a seamless and secure connection. In some embodiments, the hydrophilic coating gets pushed through using the hub. In some embodiments, an inner diameter of the hub is also coated with a hydrophilic coating as a result of this process, further reducing friction. Conventional catheters are do not include hydrophilic coating hubs as conventional manufacturing processes require catheters be coated before any hydrophilic coating is applied.

In some embodiments, the catheter includes a substantially smooth outer diameter (OD) surface. In some embodiments, the OD surface has a texture depth less than 0.002″.

In some embodiments, one or more catheters described herein include a (spiral) pattern of offset surfaces configured to enhance the lubricity of the inner diameter. In some embodiments, the contacting surface of the catheter, which interfaces with traversing devices (e.g., pusher wires, catheters) during delivery or with blood clots during aspiration, includes a width between 0.001″ and 0.004″ from one contact surface to another. In some embodiments, the contacting surface comprises materials selected from a group comprising Stainless Steel, Nitinol, Nylon, Pebax, and Pellethane as previously described.

In some embodiments, the catheter inner diameter (ID) includes a recessed surface (i.e., valley) formed between the polymer and reinforcement structure, set back from the contacting surface by a distance (i.e., depth) ranging from 0.0001″ to 0.010″. In some embodiments, the recessed surface has a width between peaks of 0.001″ and 0.007″, where the recessed surface defines the pitch of coil structure. In some embodiments, the recessed surface includes the polymer material and/or the hydrophilic coatings previously described. In some embodiments, both the contacting and recessed surfaces are coated with a hydrophilic layer(s) that may include one or more of Polyvinylpyrrolidone (PVP), Polyacrylic acid (PAA), or Hyaluronic Acid (HA), as non-limiting examples.

In some embodiments, the resulting catheter includes a hydrophilic coating with varying thickness along its length. In some embodiments, one or more contacting surfaces include a thin hydrophilic coating with a thickness from approximately 0.0001″ to 0.000198″. In some embodiments, non-contacting areas (recessed areas) include a thick hydrophilic coating ranging from 0.0002″ to 0.003″. In some embodiments, surface tension keeps the hydrophilic coating in place while curing, where more hydrophilic coating will gather in the recessed area, resulting in an inner diameter with alternating hydrophilic coating thickness.

In some embodiments, the catheter is configured to withstand vacuum forces up to 29.92″ Hg and maintain a circular shape or not collapse more than 30% of original dimension. In some embodiments, the catheter is configured to withstand a minimum burst pressure of 300 KPA (42.5 psi) (stable up to 100 psi) for a duration of 30 seconds while not leaking and/or substantially maintaining shape. In some embodiments, the catheter's construction is configured to provide tip softness and kink resistance as further described herein.

As previously mentioned, in some embodiments, the catheter includes hydrophilic-coated hubs configured to facilitate the delivery of interventional devices and/or to minimize friction during clot removal. In some embodiments, a method of identifying a hydrophilic-coated hubs includes coloring the hub with Tantalum Blue dye to enhance visibility and identification of the hydrophilic coating process.

In some embodiments, the catheter includes a hydrophilic coated inner diameter hub, which may include polycarbonate or pvacs hubs with a hydrophilic coat. In some embodiments, the coating on the interior of the hub is configured to increase lubricity, further facilitating the delivery of devices through the catheter.

In some embodiments, the catheter's hypotubes are manufactured using laser cutting techniques to create a spiral pattern. In some embodiments, the laser cuts are of a specific size and distance apart, which promotes inner lubricity. In some embodiments, the precise laser cutting technique, combined with the hydrophilic coating, provides the necessary lubricity for the catheter's inner surfaces. In some embodiments, the offset, repeating patterns in the inner diameter of the catheter enhance lubricity and allow for the elimination of a PTFE liner.

FIG. 19 shows results for a 3-point bending test performed on conventional catheters and system catheters according to some embodiments. In some embodiments, 3-point test can be used to determine flexural strength, flexural modulus (stiffness), and/or elasticity. In some embodiments, results show stiffness characteristics for a proximal section (closer to the hub) and a distal section (closer to the end) of conventional and system catheters. In some embodiments, using the system and methods described herein, a ratio between the stiffness of the distal section and the proximal section is 15% or less, which is much less than conventional catheters. In some embodiments, a distal/proximal ratio for one or more catheters described herein is between approximately 5-10%.

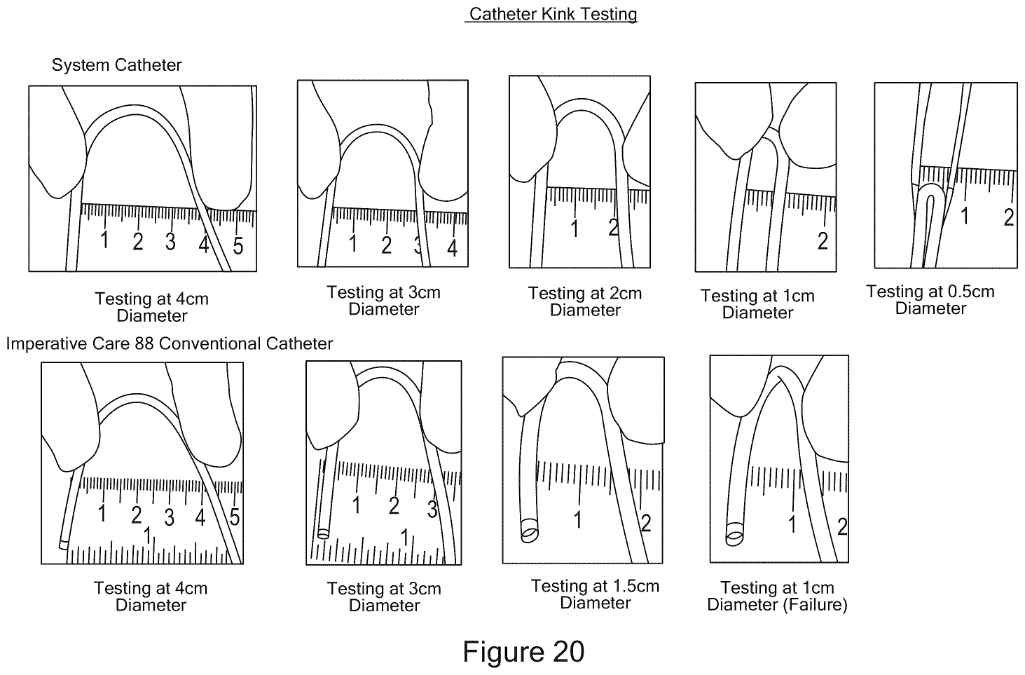

FIG. 20 illustrates a catheter kink test for a system versus a conventional catheter according to some embodiments. Kinking, in the context of this disclosure, refers to the undesirable bending, twisting, and/or creasing of a tube structure (i.e., catheter) to such an extent that it obstructs and/or blocks the motion of a fluid and/or a traversing structure moving within the tube structure. Any reference to “kinking” in this disclosure can be replaced with “collapsing” and/or “creasing” when defining the metes and bounds of the system. In addition, kinking includes a decrease in inner diameter of more than 30% along a first axis of a catheter cross-section, and/or an increase in inner diameter of more than 30% along a second axis of a catheter cross-section.

As shown in FIG. 20, conventional catheters kink and/or collapse at diameter ranges of less than 1.5 cm, where the diameter is measured from one outside portion of the catheter to the other. In contrast, in some embodiments, system catheters are configured to bend to diameter ranges less than 1.5 cm without kinking. In some embodiments, system catheters are configured to bend to diameter ranges less than 1.0 cm without kinking. In some embodiments, system catheters are configured to bend to diameter ranges less than 0.5 cm without kinking, which is far superior to any conventional catheter. In some embodiments, system catheters are configured to bend to a diameter range between 1.5 cm-5 cm.

In some embodiments, system catheters are configured to bend to an inner radius of curvature less than 16 mm without kinking. In some embodiments, system catheters are configured to bend to an inner radius of curvature less than 10 mm without kinking. In some embodiments, system catheters are configured to bend to an inner radius of curvature less than 5 mm without kinking. In some embodiments, system catheters are configured to bend to an inner radius of curvature less than 2 mm without kinking. In some embodiments, system catheters are configured to bend to an inner radius of curvature less than or equal to 1 mm without kinking, which corresponds to the 0.5 cm test shown in FIG. 20. In some embodiments, system catheters are configured to bend to an inner radius of curvature range of 15 mm-1 mm.

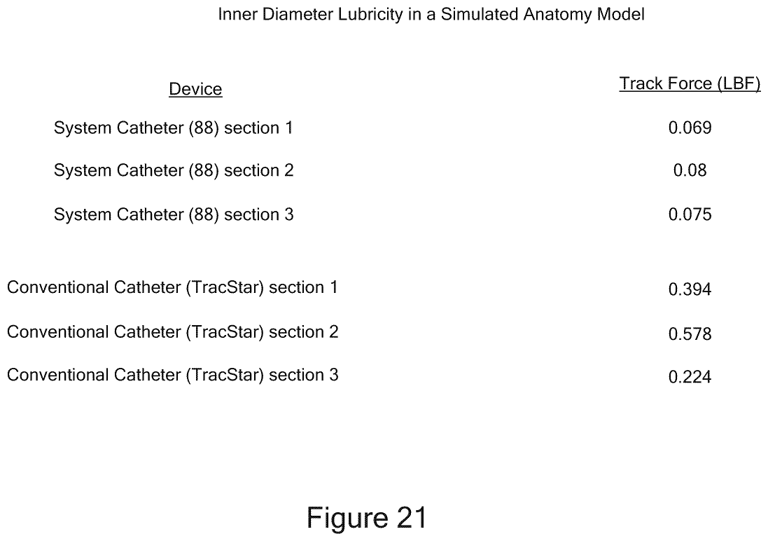

FIG. 21 shows data for inner diameter lubricity in a simulated anatomy model according to some embodiments. Inner diameter lubricity testing in a simulated anatomy model is a method used to evaluate the ease of movement (or lubricity) of devices, such as guidewires or other instruments within the lumen of a catheter, where devices need to be inserted and maneuvered through the catheter without causing damage or discomfort. In some embodiments, this test simulates the conditions that the catheter would experience in the body, including the presence of bodily fluids and the various twists and turns of the vascular system. The goal is to ensure that the catheter's inner surface is sufficiently lubricious to allow smooth, unimpeded movement of devices within it in accordance with some embodiments.

In some embodiments, the catheter is prepared by ensuring it is clean and free of any debris. In some embodiments, a lubricant, often a saline solution or a specific medical-grade lubricant, is applied to the device that will be inserted into the catheter, simulating the presence of bodily fluids. In some embodiments, a traversing structure (e.g., a guidewire) is inserted into the catheter and maneuvered through it, where the ease of movement is observed and recorded. This can be done manually or using a machine that can apply a consistent force according to some embodiments. In some embodiments, the force required to move the device through the catheter is measured using a force gauge or similar instrument. The lower the force required to move the traversing structure; the more lubricious the catheter's inner diameter is considered to be in accordance with some embodiments.

As shown in FIG. 21, in some embodiments, track forces for system catheters are significantly less than conventional catheters in similar sections. In some embodiments, a track force for system catheters is less than 0.2 lbs force (lbf) for any catheter section. In some embodiments, a track force for system catheters is less than 0.1 lbf force for any catheter section. In some embodiments, a track force for system catheters falls within a range of 0.2 lbf-0.05 lbf force for any catheter section.

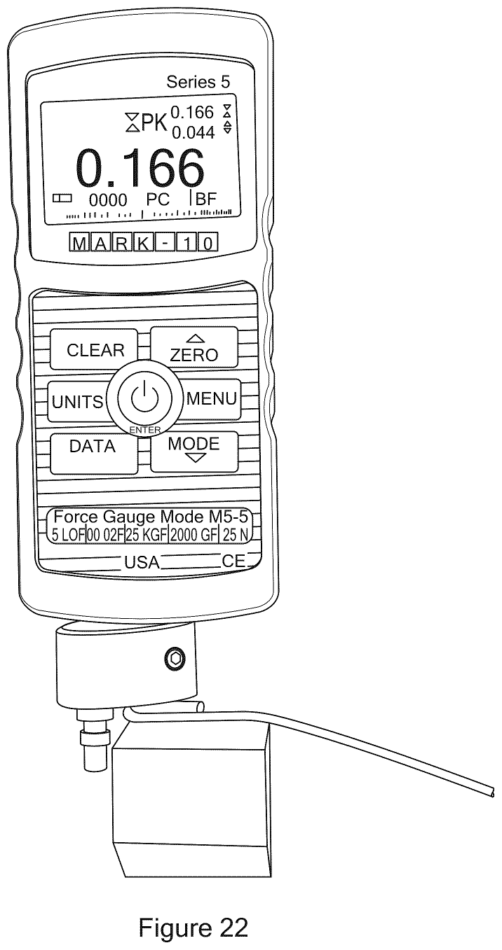

FIG. 22 shows a measured catheter distal looped compression force according to some embodiments. In some embodiments, the catheter is configured to provide a Catheter Distal Looped Compression Force of 0.009-0.300 lbf. In some embodiments, catheter tip spring back force is measured by bending the distal 2 cm to 90 degrees and measuring with a force gage the spring back force as it returns to straight. In some embodiments, spring back force of the distal end (tip) includes a range of 0.001-0.100 lbf.

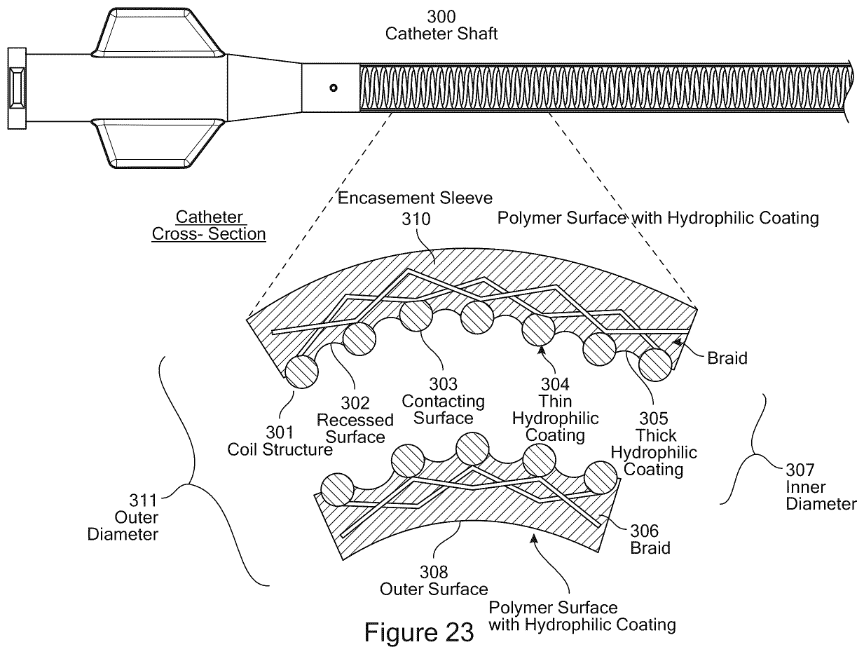

FIG. 23 shows a cross-sectional view of a catheter shaft 300, according to some embodiments. In some embodiments, the catheter shaft 300, which may be a part of any catheter described herein and/or shown in the figures, includes a coil structure 301, which is formed from one or more elements such as wire, string, or laser cut hypotube as previously described in according to some non-limiting embodiments. In some embodiments, the coil structure 301, in combination with an encasement sleeve 310, is configured to form recesses 302 along the inner diameter 307 of the catheter 300. These recessed surface 302 are set back from the contacting surface 303 in the ranges previously described according to some embodiments.

The contacting surface 303, which contacts traversing device surfaces during delivery and/or blood clots during aspiration, includes a thin hydrophilic coating 304 with a thickness from approximately 0.0001″ to 0.000198″ in some embodiments. In some embodiments, the recessed surface 302 includes a thick hydrophilic coating 305 ranging from 0.0002″ to 0.003″.

In some embodiments, the catheter 300 includes a braid 306. In some embodiments, the braid 306 is applied over the coiled structure 301. In some embodiments, only the braid 306 is used, where areas between the braid define the recessed surfaces. The braid 306, which is formed from a selected material, braid pattern, and braid picks per inch (PPI), contributes to the desired structural integrity of the catheter 300.

In some embodiments, the catheter shaft 300 includes an encasement sleeve 310 which may include one or more polymers described herein. Once the encasement sleeve 310 is coupled and/or fused to the reinforcement structure 48, which includes coil structure 301 and/or braid 306 in this non-limiting example, the combination of the reinforcement structure 48 and the encasement sleeve 310 creates peaks in valleys within the inner diameter 307 of the formed catheter shaft 300. In some embodiments, as the encasement sleeve 310 is deformed during the coupling process, at least part of the encasement sleeve 310 flows through the hollow portions formed by the reinforcement structure 48. In some embodiments, once coupled, the catheter shaft 300 includes a smooth outer surface 308 defining the limits of the outer diameter 311 which is smoother than the surface of the inner diameter 307, wherein each surface is coated with at least one hydrophilic coating, enhancing the catheter's lubricity and ease of use during medical procedures.

None of the steps described herein are essential or indispensable. Any of the steps according to some embodiments can be adjusted or modified. In some embodiments, other or additional steps can be used. Any portion of any of the steps, processes, structures, and/or devices disclosed or illustrated in some embodiments, flowcharts, or examples in this specification can be combined or used with or instead of any other portion of any of the steps, processes, structures, and/or devices disclosed or illustrated in some embodiments, flowcharts, or examples. Some embodiments and examples provided herein are not intended to be discrete and separate from each other.

The section headings and subheadings provided herein are nonlimiting. The section headings and subheadings do not represent or limit the full scope of some embodiments described in the sections to which the headings and subheadings pertain. For example, a section titled “Topic 1” may include embodiments that do not pertain to Topic 1 and some embodiments described in other sections may apply to and be combined with embodiments described within the “Topic 1” section.

The various features and processes described above may be used independently of one another, or may be combined in various ways. All possible combinations and subcombinations according to some embodiments are intended to fall within the scope of this disclosure. In some embodiments, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events according to some embodiments may be performed in an order other than the order specifically disclosed. In some embodiments, multiple steps may be combined in a single block or state. In some embodiments, the example tasks or events may be performed in serial, in parallel, or in some other manner. Tasks or events according to some embodiments may be added to or removed from the disclosed example embodiments. In some embodiments, the example systems and components described herein may be configured differently than described. For example, elements may be added to, removed from, or rearranged compared to the disclosed example according to embodiments.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function according to some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. While certain example embodiments have been described, these embodiments have been presented by way of example only, and are not intended to limit the scope of the system disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the system disclosed herein.

I claim:

1. A catheter comprising:
an inner diameter,
a reinforcement structure,
an encasement sleeve, and
an outer diameter;
wherein the reinforcement structure is configured to at least partially form a shape of the inner diameter along a length of the catheter;
wherein the encasement sleeve is configured to encase at least a portion of the reinforcement structure; and
wherein the encasement sleeve is configured to prevent a fluid flow from the inner diameter of the catheter to the outer diameter of the catheter.

2. The catheter of claim 1,
wherein the reinforcement structure includes gaps between reinforcement surfaces along the inner diameter; and
wherein the reinforcement surfaces include the portion of the reinforcement structure orthogonal to a center point of the inner diameter.

3. The catheter of claim 2,
wherein the reinforcement structure is configured to define one or more shapes along the inner diameter of the catheter.

4. The catheter of claim 1,
wherein an inner diameter varies along at least a portion of the length of the inner diameter.

5. The catheter of claim 1,
wherein a distance between the inner diameter and the outer diameter varies along at least a portion of the length of the catheter.

6. The catheter of claim 1,
wherein the catheter does not comprise Polytetrafluoroethylene.

7. The catheter of claim 1,
wherein the catheter does not comprise a Polytetrafluoroethylene liner along the inner diameter.

8. The catheter of claim 1,
wherein an outer diameter surface varies less than an inner diameter surface.

9. The catheter of claim 1,
wherein an outer diameter surface is smoother than an inner diameter surface.

10. The catheter of claim 1,
wherein the reinforcement structure includes a coil structure.

11. The catheter of claim 1,
further including a hub;
wherein an inner diameter of the hub is coated with a hydrophilic substance.

12. A method of manufacturing a catheter comprising steps of:
forming or placing a reinforcement structure on a mandrel;
placing at least one polymer over the reinforcement structure;
coupling the reinforcement structure and the at least one polymer together to create a catheter shaft; and
removing the catheter shaft from the mandrel.

13. The method of claim 12,
wherein an outer surface of the catheter shaft is smoother than an inner surface of the catheter shaft after it is removed from the mandrel.

14. The method of claim 13,
wherein the inner surface includes peaks formed by the reinforcement structure; and
wherein the inner surface includes valleys formed by the at least one polymer.

15. The method of claim 13,
wherein the reinforcement structure includes hollow portions; and
wherein valleys are formed by a portion of the at least one polymer exposed between the hollow portions.

16. The method of claim 12, further comprising steps of:
coupling a hub to a proximal end of the catheter shaft.

17. The method of claim 16, further comprising steps of:
coating an inner diameter of the hub with a hydrophilic substance.

18. The method of claim 16, further comprising steps of:
coating an inner diameter of the catheter shaft and an inner diameter of the hub with a hydrophilic substance.

19. The method of claim 16,
wherein the reinforcement structure includes hollow portions configured to impart flexibility to the catheter shaft; and
wherein a distal 3-point bend force of the catheter shaft at a distal region furthest from a hub attachment area is 15% or less of a proximal 3-point bend force required to bend the catheter shaft at a proximal region closest from the hub.

20. The method of claim 13,
wherein at least a portion of the catheter shaft is configured to bend to an inner radius of curvature less than 1 cm without kinking.
